(19) United States
(12) Patent Application Publication
(10) Pub. No.: US 2012/0059232 A1
(43) Pub. Date: Mar. 8, 2012

(54) IMPLANTABLE OPTICAL GLUCOSE SENSING

(75) Inventors: Yossi Gross, Mazor (IL); Tehila Hyman, Modi'in (IL); Tamir Gil, Givat Haim (Meuchad) (IL)

(73) Assignee: GLUSENSE, LTD., Lod (IL)

(21) Appl. No.: 13/141,936

(22) PCT Filed: Dec. 24, 2009
(86) PCT No.: PCT/IL09/01214

§ 371 Date: Nov. 14, 2011

Related U.S. Application Data
(63) Continuation-in-part of application No. 12/344,103, filed on Dec. 24, 2008.
(60) Provisional application No. 61/149,110, filed on Feb. 2, 2009.

(51) Int. Cl. A61B 5/1455

(52) U.S. Cl. 600/316; 600/310

(57) ABSTRACT

Apparatus is provided, including a support (21) configured to be implanted within a body of a subject and a sampling region (30, 1430) coupled to the support (21). The apparatus is configured to passively allow passage through the sampling region (30, 1430) of at least a portion of fluid from the subject. The apparatus also includes an optical measuring device in optical communication with the sampling region (30, 1430). The optical measuring device comprises at least one light source (40) configured to transmit light through at least a portion of the fluid, and at least one sensor (42) configured to measure a parameter of the fluid by detecting light passing through the fluid. Other applications are also described.

### CROSS-REFERENCES TO RELATED APPLICATIONS

The present application:

(a) is a continuation-in-part of and claims the priority from U.S. patent application Ser. No. 12/344,103 to Gross et al., entitled, “Implantable optical glucose sensing,” filed Dec. 24, 2008;

(b) claims the priority from U.S. Provisional Patent Application 61/149,110 to Gil et al., entitled, “Compact optical sensor for flat fluorescent sample regions,” filed Feb. 2, 2009; and

(c) is related to:
U.S. Provisional Patent Application 60/588,211 to Gross et al., entitled, “Implantable sensor,” filed Jul. 14, 2004;
U.S. Provisional Patent Application 60/658,716 to Gross et al., entitled, “Implantable fuel cell,” filed Mar. 3, 2005;
PCT Patent Application PCT/IL2005/000743 to Gross et al., entitled “Implantable power sources and sensors,” filed Jul. 13, 2005;
U.S. Provisional Patent Application 60/786,532 to Gross et al., entitled, “Implantable sensor,” filed Mar. 28, 2006; and
PCT Patent Application PCT/IL2007/000399 to Gross, entitled “Implantable sensor,” filed Mar. 28, 2007.

All of these applications are incorporated herein by reference.

### FIELD OF THE INVENTION

Some applications of the present invention relate generally to implantable sensors and specifically to methods and apparatus for sensing blood glucose concentrations.

### BACKGROUND

Diabetes mellitus is a disease in which cells fail to uptake glucose either due to a lack of insulin (Type I) or an insensitivity to insulin (Type II). The associated elevation of blood glucose levels for prolonged periods of time has been linked to a number of problems including retinopathy, nephropathy, neuropathy, and heart disease. A typical care regimen for Type I diabetics includes daily monitoring of blood glucose levels and injection of an appropriate dose of insulin. Conventional glucose monitoring involves the use of an invasive “finger-stick” method in which the finger of a subject is pricked in order to withdraw a small amount of blood for testing in a diabetes monitoring kit based on the electroenzymatic oxidation of glucose.

Polarimetric measurement of glucose concentration is based on optical rotatory dispersion (ORD) a phenomenon by which a solution containing a chiral molecule rotates the plane of polarization for linearly polarized light passing through it. The rotation is the result of a difference in refractive indices nL and nR for left and right circularly polarized light traveling through the electron cloud of a molecule.

Fluorescence is a photochemical phenomenon in which a photon of specific light wavelength (excitation wavelength) strikes an indicator (fluorophore) molecule, thereby exciting an electron to a higher energy state. As that “excited” electron decays back down to its original ground state, another photon of light is released at a longer wavelength (emission wavelength).

Fluorescence resonance energy transfer (FRET) involves the transfer of non-photonic energy from an excited fluorophore (the donor) to another fluorophore (the acceptor) when the donor and acceptor molecules are in close proximity to each other. FRET enables the determination of the relative proximity of the molecules for investigating, for example, molecular interactions between two protein partners, structural changes within one molecule, ion concentrations, and the binding of analytes.

PCT Publication WO 07/110,867 to Gross and Hyman, which is incorporated herein by reference, describes an implanted apparatus for detecting the concentration of a substance (e.g., blood glucose) in a subject. The apparatus includes a housing adapted to be implanted in the subject, the housing including an optical detector, light source, and living cells that are genetically engineered to produce, in a patient's body, a sensor protein that is able to bind with an analyte and to undergo a conformational change in a detectable manner. Typically, but not necessarily, FRET techniques are used to detect the conformational change. In a typical case described therein, there is provided a cell capable of producing a protein that includes an analyte binding protein, a donor fluorescent protein, and an acceptor fluorescent protein. The protein is configured such that binding of the analyte causes a change in the distance between said donor and said acceptor proteins, respectively. This change in distance between the donor and the acceptor proteins changes the amount of non-photonic energy that is transferred from the donor to the acceptor. Thus, upon excitation of the donor proteins, there is a change in the ratio between the intensity of fluorescent light that is emitted by donor proteins and the intensity of fluorescent light that is emitted by acceptor proteins.

Fluorescent proteins (FP) pairs that are useful for performing FRET measurements in living cells include the cyan fluorescent protein (CFP) as the donor and the yellow fluorescent protein (YFP) as the acceptor, because the emission spectrum of CFP only partially overlaps the excitation spectrum of YFP. Accordingly, in some applications of PCT Publication WO 07/110,867 to Gross and Hyman, the analyte is glucose, the binding protein is a glucose binding protein (GBP), the donor is CFP, and the acceptor is YFP.

US Patent Application Publication 2007-0066877 to Arnold et al. describes an implantable microspectrometer for the reagentless optical detection of an analyte in a sample fluid. The microspectrometer comprises an optical sampling cell having a cell housing defining a fluid inlet port and a fluid outlet port, the fluid inlet port configured to receive an optical sampling fluid from a test subject; an electromagnetic radiation source in communication with a first portion of the optical sampling cell housing and configured to irradiate at least a portion of the optical sampling fluid with electromagnetic radiation; and an electromagnetic radiation detector in communication with a second portion of the optical sampling cell housing and configured to detect electromagnetic radiation emanating from the optical sampling cell. In use, the implantable microspectrometer can optically detect at least one parameter of an analyte contained within the optical sampling fluid in the absence of an added reagent.

U.S. Pat. No. 6,049,727 to Crothall describes an in vivo implantable sensor which obtains spectra of body fluid constituents and processes the spectra to determine the concentration of a constituent of the body fluid. The sensor includes an optical source and detector. The source emits light at a plurality of different, discrete wavelengths, including at least one wavelength in the infrared region. The light interacts with the body fluid and is received at the detector. The light at the plurality of different wavelengths has a substantially collinear optical path through the fluid with respect to each other. When measuring fluid constituents in a blood vessel, such as blood glucose, the light at the plurality of different wavelengths is emitted in a substantially single period of time. The spectra are corrected for artifacts introduced from extraneous tissue in the optical path between the source and the detector. The sensor is fully implanted and is set in place to allow plural measurements to be taken at different time periods from a single in vivo position. The light source emits at least three different wavelengths.

U.S. Pat. No. 6,577,393 to Potzschke et al. describes a process or device for determination of the plane of polarization of polarized light. Light from a light source is polarized by means of a polarizing filter which has a certain setting angle .theta.sub.0 with respect to the first reference plane, the plane of incidence on the reflecting surface. The polarized beam passes through the sample in the measurement chamber, in which the angle of rotation is changed by the small angle .theta.sub.MG. The sum of .theta.sub.0 and .theta.sub.MG gives the angle of rotation .theta.sub.e, at which the beam emerging from the measurement chamber is partially reflected at the surface of a medium of higher refractive index. The reflected beam is then separated into two partial beams (an extraordinary beam and an ordinary beam), with vibration directions exactly perpendicular to each other. In a polarizing prism, the reference plane of which, the plane of vibration of the ordinary beam, has a certain setting angle (.theta.*) with respect to the first reference plane. The intensities I.sub.o and I.sub.a of the two partial beams are determined photometrically by detectors and the ratio of the measured intensities is determined by a quotient determiner.

U.S. Pat. No. 5,209,231 to Cote et al. describes optically based apparatus for non-invasively determining the concentration of optically active substances in a specimen. The apparatus comprises a source of a beam of spatially coherent light which is acted upon to produce a rotating linear polarized vector therein. A beam splitter splits the beam into a reference beam and a detector beam for passage through the specimen. The detector beam is received upon exiting the specimen and compared with the reference beam to determine the amount of phase shift produced by passage through the specimen. This amount of phase shift is converted into concentration of the optically active substance in the specimen.

U.S. Pat. No. 6,188,477 to Pu et al. describes integrated polarization sensing apparatus and method uses a self-homodyne detection scheme to provide required sensitivity for numerous applications, such as glucose concentration monitoring, without the need for expensive, bulky components. The detection scheme is implemented by splitting a polarized laser beam with a polarization beam splitter into a P wave component and an S wave component, phase modulating the P wave component and recombining the two components. The polarization of the combined optical beam is then rotated slightly by the variable to be monitored, such as by passing the beam through a glucose solution. Finally, the beam is passed onto a photodetector that generates a signal that is proportional to the polarization rotation angle. This device has the advantage of employing optical components, including polarizing beam splitters, phase modulators and lenses, that can all be fabricated on a single silicon chip using MEMS technology so that the device can be made compact and inexpensive.

U.S. Pat. No. 6,061,582 to Small et al., describes non-invasive measurements of physiological chemicals such as glucose in a test subject using infrared radiation and a signal processing system. The level of a selected physiological chemical in the test subject is determined in a non-invasive and quantitative manner by a method comprising the steps of: (a) irradiating a portion of the test subject with near infrared radiation; (b) collecting data concerning the irradiated light on the test subject; (c) digitally filtering the collected data to isolate a portion of the data indicative of the physiological chemical; and (d) determining the amount of physiological chemical in the test subject by applying a defined mathematical model to the digitally filtered data. The collected data is in the form of either an absorbance spectrum or an interferogram.

U.S. Pat. No. 6,587,704 to Fine et al., describes a non-invasive method of optical measurements for determining at least one desired parameter of patient's blood. The method utilizes reference data indicative of the values of the desired blood parameter as a function of at least two measurable parameters. At least one of the measurable parameters is derived from scattering spectral features of the medium highly sensitive to patient individuality, and the at least one other measurable parameter is indicative of artificial kinetics of the optical characteristics of the patient's blood perfused fleshy medium. A condition of artificial kinetics is created at a measurement location, and maintained during a certain time. Measurements are carried out with different wavelengths of incident light during a time period including this certain time. Measured data is in the form of time evolutions of light responses of the medium corresponding to the different wavelengths. By analyzing the measured data, the at least two measurable parameters are extracted, and the reference data is utilized to determine the at least one desired blood parameter.

US Patent Application Publication 2007-0004974 to Nagar et al., describes apparatus for assaying an analyte in a body comprising: at least one light source implanted in the body controllable to illuminate a tissue region in the body with light at least one wavelength that is absorbed by the analyte and as a result generates photoacoustic waves in the tissue region; at least one acoustic sensing transducer coupled to the body that receives acoustic energy from the photoacoustic waves and generates signals responsive thereto; and a processor that receives the signals and processes them to determine a concentration of the analyte in the illuminated tissue region.

U.S. Pat. No. 3,837,339 to Aisenberg et al., describes techniques for monitoring blood glucose levels, including an implantable glucose diffusion-limited fuel cell. The output current of the fuel cell is proportional to the glucose concentration of the body fluid electrolyte and is therefore directly indicative of the blood glucose level. This information is telemetered to an external receiver which generates an alarm signal whenever the fuel cell output current exceeds or falls below a predetermined current magnitude which represents a normal blood glucose level. Valve means are actuated in response to the telemetered information to supply glucose or insulin to the monitored living body.

U.S. Pat. Nos. 5,368,028 and 5,101,814 to Palti, describe methods and apparatus for monitoring blood glucose levels by implanting glucose sensitive living cells, which are enclosed in a membrane permeable to glucose but impermeable to immune system cells, inside a patient. Cells that produce detectable electrical activity in response to changes in blood glucose levels are used in the apparatus along with sensors for detecting the electrical signals, as a means for detecting blood glucose levels. Human beta cells from the islets of Langerhans of the pancreas, sensor cells in taste buds, and alpha cells from the pancreas are discussed as appropriate glucose sensitive cells.

Methods for immunoprotection of biological materials by encapsulation are described, for example, in U.S. Pat. Nos. 4,352,883, 5,427,935, 5,879,709, 5,902,745, and 5,912,005. The encapsulating material is typically selected so as to be biocompatible and to allow diffusion of small molecules between the cells of the environment while shielding the cells from immunoglobulins and cells of the immune system. Encapsulated beta cells, for example, can be injected into a vein (in which case they will eventually become lodged in the liver) or embedded under the skin, in the abdominal cavity, or in other locations. Fibrotic overgrowth around the implanted cells, however, gradually impairs substance exchange between the cells and their environment. Hypoxia of the cells typically leads to cell death.

PCT Patent Publication WO 2006/006166 to Gross et al., which is incorporated herein by reference, describes a protein, including a glucose binding site, cyan fluorescent protein (CFP), and yellow fluorescent protein (YFP). The protein is described as being configured such that binding of glucose to the glucose binding site causes a reduction in a distance between the CFP and the YFP. Apparatus is also described for detecting a concentration of a substance in a subject, the apparatus comprising a housing adapted to be implanted in the subject. The housing comprises an optical detector, and cells genetically engineered to produce, in a patient's body, a FRET protein comprising a fluorescent protein donor, a fluorescent protein acceptor, and a protein containing a binding site for the substance.

United States Patent Application Publication 2005/0118726 to Schultz et al. describes a method for making a fusion protein, having a first binding moiety having a binding domain specific for a class of analytes that undergoes a reproducible allosteric change in conformation when said analytes are reversibly bound; a second moiety and third moiety that are covalently linked to either side of the first binding moiety such that the second and third moieties undergo a change in relative position when an analyte of interest molecule binds to the binding moiety; and the second and third moieties undergo a change in optical properties when their relative positions change and that change can be monitored remotely by optical means. A system and method is also described for detecting glucose that uses such a fusion protein in a variety of formats including subcutaneously and in a bioreactor.

U.S. Pat. No. 5,998,204 to Tsien et al. describes fluorescent protein sensors for detection of analytes. Fluorescent indicators including a binding protein moiety, a donor fluorescent protein moiety, and an acceptor fluorescent protein moiety are described. The binding protein moiety has an analyte-binding region which binds an analyte and causes the indicator to change conformation upon exposure to the analyte. The donor moiety and the acceptor moiety change position relative to each other when the analyte binds to the analyte-binding region. The donor moiety and the acceptor moiety exhibit fluorescence resonance energy transfer when the donor moiety is excited and the distance between the donor moiety and the acceptor moiety is small. The indicators can be used to measure analyte concentrations in samples, such as calcium ion concentrations in cells.

An article by Olesberg J T et al., “Optical microsensor for continuous glucose measurements in interstitial fluid,” Optical Diagnostics and Sensing VI, Proc. of SPIE Vol. 6094, 609403, pp. 1605-7422 (2006), describes an optical glucose microsensor based on absorption spectroscopy in interstitial fluid that can potentially be implanted to provide continuous glucose readings. Light from a GaInAsSb LED in the 2.2-2.4 urn wavelength range is passed through a sample of interstitial fluid and a linear tunable filter before being detected by an uncooled, 32-element GaInAsSb detector array: Spectral resolution is provided by the linear tunable filter, which has a 10 nm band pass and a center wavelength that varies from 2.18-2.38 um (4600-4200 cm̂−1) over the length of the detector array. The sensor assembly is a monolithic design requiring no coupling optics. In the present system, the LED running with 100 mA of drive current delivers 20 nW of power to each of the detector pixels, which have a noise-equivalent-power of 3 pW/Hẑ(½). This is sufficient to provide a signal-to-noise ratio of 4500 Hẑ(½) under detector-noise limited conditions. This signal-to-noise ratio corresponds to a spectral noise level less than 10 uAU for a five minute integration, which is described as being sufficient for sub-millimolar glucose detection.

An article by Klueh U. et al., entitled, “Enhancement of implantable glucose sensor function in vivo using gene transfer-induced neovascularization,” Biomaterials, April, 2005, 26(10):1155-63, states that the in vivo failure of implantable glucose sensors is thought to be largely the result of inflammation and fibrosis-induced vessel regression at sites of sensor implantation. To determine whether increased vessel density at sites of sensor implantation would enhance sensor function, cells genetically engineered to over-express the angiogenic factor (AF) vascular endothelial cell growth factor (VEGF) were incorporated into an ex ova chicken embryo chorioallantoic membrane (CAM)-glucose sensor model. The VEGF-producing cells were delivered to sites of glucose sensor implantation on the CAM using a tissue-interactive fibrin bio-hydrogel as a cell support and activation matrix. This VEGF-cell-fibrin system induced significant neovascularization surrounding the implanted sensor, and significantly enhanced the glucose sensor function in vivo.

The following patents and patent applications may be of interest:
PCT Publication WO 01/50983 to Bloch et al.
PCT Publication WO 01/50983 to Vardi et al., and U.S. patent application Ser. No. 10/466,069 in the national phase thereof.
PCT Publication WO 04/028358 to Caduff et al.
PCT Publication WO 04/089465 to Penner et al.
PCT Publication WO 05/053523 to Caduff et al.
PCT Publication WO 06/097933 to Bitton et al.
PCT Publication WO 08/018,079 to Goldberg et al.
PCT Publication WO 90/15526 to Kertz
U.S. Pat. No. 4,402,694 to Ash et al.
U.S. Pat. Nos. 4,981,779 and 5,001,054 to Wagner
U.S. Pat. No. 5,011,472 to Aebischer et al.
U.S. Pat. No. 5,089,697 to Prohaska
U.S. Pat. No. 5,116,494 to Chick et al.
U.S. Pat. No. 5,443,508 to Giampapa
U.S. Pat. No. 5,529,066 to Palti
U.S. Pat. No. 5,614,378 to Yang et al.
U.S. Pat. No. 5,702,444 to Struthers et al.
U.S. Pat. No. 5,741,334 to Mullon et al.
U.S. Pat. No. 5,834,005 to Usala
U.S. Pat. No. 5,855,613 to Antanavich et al.
U.S. Pat. Nos. 5,894,351; 5,910,661; 5,917,605; 6,304,766; 6,330,464; 6,711,423; 6,940,590; 7,016,714; 7,135,342; 7,157,723; 7,190,445; 7,227,156; 7,308,292; 7,375,347; and 7,405,387 to Colvin Jr., et al.
U.S. Pat. No. 6,091,974 to Palti
U.S. Pat. No. 6,400,974 to Lesho
U.S. Pat. No. 6,630,154 to Fraker et al.
U.S. Pat. No. 6,671,527 to Petersson et al.
U.S. Pat. No. 6,846,288 to Nagar et al.
U.S. Pat. No. 7,068,867 to Adoram et al.
U.S. Pat. No. 7,184,810 to Caduff et al.
U.S. Pat. No. 7,228,159 to Petersson et al.
US Patent Application 2002/0038083 to Houben and Larik
United States Patent Application Publication 2003/0232370 to Trifiro
US Patent Application Publication 2003/0134346 to Amiss et al.

The following articles may be of interest:
Patounakis G et al. “Active CMOS Array Sensor for Time-Resolved Fluorescence Detection,” IEEE Journal of Solid-state Circuits 41(11):2521-30 (2006)
Yu-Lung L et al., “A polarimetric glucose sensor using a liquid-crystal polarization modulator driven by a sinusoidal signal,” Optics Communications 259(1), pp. 40-48 (2006)
McNichols J et al., “Development of a non-invasive polarimetric glucose sensor,” IEEE-LEOS Newsletter, 12:30-31 (1998)
Cote G L “Noninvasive and minimally-invasive optical monitoring technologies,” The Journal of Nutrition 131:1596 S-1604S (2001)
Wan Q, “Dual wavelength polarimetry for monitoring glucose in the presence of varying birefringence,” A thesis submitted to the Office of Graduate Studies of Texas A&M University (2004)
Olesberg J T, “Noninvasive blood glucose monitoring in the 2.0-2.5 μm wavelength range,” Lasers and Electro-Optics Society. LEOS 2001. The 14th Annual Meeting of the IEEE. Volume: 2, p. 529
Olesberg J T et al., “Tunable Laser Diode System for Noninvasive Blood Glucose Measurements,” Appl. Spectrosc. 59, pp. 1480-1484 (2005)
Olesberg J T et al., “In vivo near-infrared spectroscopy of rat skin tissue with varying blood glucose levels,” Analytical Chemistry 78, pp. 215-223 (2006)
Amir O et al., “Accurate home and clinical use of a non-invasive continuous glucose monitor,” (2006)
Dvir D et al., “Non invasive blood glucose monitoring in the critically ill patients,” European Society for Clinical Nutrition and Metabolism Congress, Istanbul (2006)
Primack H, “Non-invasive sensing of glucose and hemoglobin,” Optical Imaging (2006)
Amir O et al., “Evaluation of a non-invasive continuous glucose monitoring device in a home use setting,” European Association for the Study of Diabetes, 42nd Annual Meeting, Copenhagen-Malmoe, Denmark-Sweden (2006)
Amir O et al., “Highly accurate non-invasive continuous glucose monitoring in clinical and home use settings,” American Diabetes Association, 66th Scientific Session, Washington, D.C. (2006)
Berrebi A et al., “A non-invasive evaluation of hematocrit with a new optical sensor,” European Hematology Association, 11th Congress, Amstaerdam (206)
Kononenko A et al., “Evaluation of a non-invasive blood glucose monitoring device for critically ill patients,” 26th International Symposium on Intensive Care and Emergency Medicine, Brussels (2006)
Primack H, “Non-invasive optical sensing of blood hemoglobin and glucose,” Photonic West, San Jose, Calif. (2006)
Ye K et al., “Genetic engineering of an allosterically based glucose indicator protein for continuous glucose monitoring by fluorescence resonance energy transfer,” Analytical Chemistry, 2003, 75(14), 3451-3459
Tolosa L et al., “Glucose sensor for low-cost lifetime-based sensing using a genetically engineered protein,” Analytical Biochemistry, 267, 114-120 (1999)
Helling a H et al., “Protein engineering and the development of generic biosensors,” Tibtech, Volume 16 (April, 1998)
Fehr M et al., “In vivo imaging of the dynamics of glucose uptake in the cytosol of COS-7 cells by fluorescent nanosensors,” J. Biol. Chem., 278(21):19127-19133 (2003)
Fehr M et al., “Minimally invasive dynamic imaging of ions and metabolites in living cells,” Curr Opin Plant Biol 7(3):345-51 (2004)
Laxman B et al., “Noninvasive real-time imaging of apoptosis,” Proc Natl Acad Sci USA 99(26):16551-5 (2002)
Serganova I et al., “Reporter gene imaging: potential impact on therapy,” Nucl Med Biol 32(7):763-80 (2005)
Deuschle K et al., “Genetically encoded sensors for metabolites,” Cytometry A 64(1):3-9 (2005)
Pickup J C et al., “In vivo glucose monitoring: the clinical reality and the promise,” Biosens Bioelectron 20(10):1897-902 (2005)
Ackland-Berglund, C et al., “Efficacy of tetracycline-controlled gene expression is influenced by cell type,” BioTechniques 18, 196-200 (1995)
Fillat C et al., “Suicide gene therapy mediated by the herpes simplex virus thymidine kinase gene/ganciclovir system: Fifteen years of application,” Current Gene Therapy, 3(1), pp. 13-26, (February, 2003)
Scognamiglio V et al., “Protein-based biosensors for diabetic patients,” Journal of Fluorescence, 14(5), 491-498 (September, 2004)
Moschou E et al., “Fluorescence glucose detection: Advances toward the ideal in vivo biosensor,” Journal of Fluorescence, 14(5), 535-547 (September, 2004)
Reszka R et al., “Liposome-mediated suicide gene therapy in humans,” Methods in Enzymology, 391, 200-208 (2005)
Deuschle K et al., “Construction and optimization of a family of genetically encoded metabolite sensors by semirational protein engineering,” Protein Sci. 14: 2304-2314 (2005)
Liva L et al., “Glucose permeable poly (dimethyl siloxane) poly (N-isopropyl acrylamide) interpenetrating networks as ophthalmic biomaterials,” Biomaterials Volume 26, Issue 3 pp. 233-244 (2005)
Yonzon C R et al., “A glucose biosensor based on surface-enhanced Raman scattering: Improved partition layer, temporal stability, reversibility, and resistance to serum protein interference,” Anal. Chem., 76 (1), pp. 78-85 (2004)
Koo T W et al., “Measurement of glucose in human blood serum using Raman spectroscopy”, IEEE-LEOS Newsletter 12(2) 18 (1998).
Yokota M et al., “A compact polarimetric glucose sensor using a high-performance fibre-optic Faraday rotator,” Meas. Sci. Technol. 15 pp. 143-147 (2004)

### SUMMARY OF THE INVENTION

In some applications of the present invention, a support, e.g., a housing or a scaffold, is configured to be implanted within a body of a subject and is coupled to (a) a sampling region, e.g., a chamber, configured to passively allow passage therethrough of a fluid from a subject, and (b) an optical measuring device which measures a parameter of the fluid in the chamber. Typically, the housing is subcutaneously implanted within the subject. Typically, the optical measuring device is configured to measure a concentration of an analyte, e.g., glucose, in interstitial fluid of the subject. The optical measuring device typically comprises a light source, i.e., a system providing visible or non-visible light, and a detecting system. The support provides a sampling region which is typically disposed in optical communication with the light source and the detecting system.

In some applications of the present invention, the support comprises and/or is coupled to an optically-transparent and glucose-permeable material, e.g., a gel or polymer, configured to define the sampling region of the support.

Alternatively or additionally, the sampling region is surrounded by a selectively-permeable membrane which restricts passage therethrough of substances, e.g., cells, which could potentially interfere with the measuring of the parameter of the fluid. The membrane may be present in the support independently of or in combination with the transparent glucose-permeable material. Typically, the membrane is configured to restrict passage, into the sampling region, of cells and molecules having a molecular weight greater than the molecular weight of the analyte configured to be measured by the device. In some applications of the present invention, the sampling region comprises cells engineered to produce a protein that is able to bind with the analyte and to undergo a conformational change in a detectable manner. An implanted device detects the conformational change, and, in response, generates a signal indicative of a level of the analyte in the subject. Typically, but not necessarily, FRET techniques known in the art are used to detect the conformational change. These genetically-engineered cells may be used in combination with the detection methods described hereinbelow.

The concentration of the analyte is measured using polarimetric techniques which measure the concentration of the analyte according to the polarization of light that passes from the light source and through the sampling region. In such an application, polarizing filters are disposed in optical communication with the light source and/or the detecting system. Alternatively or additionally, the concentration of the analyte is measured using absorbance spectroscopy techniques. In such an application, the absorbance spectroscopy is used to directly measure the concentration of the analyte in the sampling region. Alternatively or additionally, the absorbance spectroscopy device comprises a plurality of detectors configured to detect optical scattering of the illuminated light (the scattering being induced by the presence of the analyte in the fluid). The plurality of detectors are configured to increase the signal-to-noise ratio.

Techniques described herein to optically measure the concentration of the analyte in the fluid typically use LEDs, solid-state lasers, or laser diodes as the light source, and photodetectors, e.g., linear detector arrays, as the detecting system.

In some applications of the present invention, the device comprises at least one mirror disposed in the optical path between the light source and the detecting system. The mirror is configured to lengthen the optical path of the light emitted from the light source.

In some applications of the present invention, the support comprises an annular ring scaffold having a wall thereof. The annular ring defines a substantially disc-shaped sampling region and houses a plurality of light sources and detecting systems. For some applications of the present invention, the annular ring has an upper surface and a lower surface that are each coupled to a respective selectively-permeable membrane which restricts passage of cells into the sampling region. In some applications of the present invention, the scaffold houses the glucose-permeable and optically-transparent material in combination with the respective membranes. Alternatively, the selectively-permeable membranes are not provided, and the glucose-permeable and optically-transparent material generally provides the functionality of the membranes. Typically, the upper surface and/or the lower surface of the disc-shaped sampling region provide a large surface area for passive fluid transport through the sampling region. Typically, the combined surface area provided for substance transport by the upper and lower surfaces of the sampling region is at least 50% (e.g., at least 70%) of a total surface area of the optical measuring device.

In some applications of the present invention, the sampling region is disposed remotely from the optical measuring device. For example, the sampling region may be disposed within a blood vessel, e.g., a vena cava, of the subject, while the optical measuring device is disposed outside of the blood vessel. In some applications of the present invention, the optical measuring device is disposed outside the body of the subject. Alternatively, the optical measuring device is disposed inside the body of the subject in a vicinity of the blood vessel and is coupled to the sampling region by optical fibers. In such an application, the optical measuring device measures a concentration of an analyte in the blood of the subject.

Typically, the implantable sensor devices described herein are configured for detection of electromagnetic emissions from an indicator having an analyte of interest permeating therethrough, wherein the characteristics of the emissions vary as a function of the concentration of the analyte. More particularly, some applications of the present invention relate to fluorescent indicators that are optically excited and are shaped to define a large interface to the surrounding body tissue and/or fluid. This large interface provides a short transfer path for the analyte from the surrounding body tissue and/or fluid to contact with the indicator that is disposed in the sampling region.

For the purpose of performing typical FRET light having at least one excitation wavelength band is transmitted in order to excite the donor molecules of a FRET-based molecule, and light having at least two wavelength bands is used for detecting the fluorescent emitted light from the donor and from the acceptor. However, in complex systems and certain environments, different factors may interfere with the pure FRET-induced signals when performing the measurements. Among these interferences are typical measurement noise issues and biological diversity that leads to varying background signals. Increasing the number of light source units and light detectors typically enhances system sensitivity and signal-to-noise ratio. Moreover, adding controlled variation of the spectral bands of both illumination and detection increases the number of independent equations that serve for data analysis thus improving the possibility of extracting the signal of interest from noise. In different applications of the present invention disclosed herein, several light source units and photo-detectors are combined into a single compact device.

For some applications of the present invention, an implantable device is provided having a substantially flat analyte measuring site, or sampling region, comprising a reacting material which facilitates analyte measurement. The device facilitates diffusion of analytes as well as diffusion of other factors (e.g., nutrients for applications in which the implantable device houses living cells) through the sampling region. The spatial configuration of such a device: (1) supports a short transfer distance from an external surface of the device into the reacting material disposed at its measurement site, or sampling region, and (2) provides a large interface area for transfer of factors from the external surroundings and into the analyte measuring site. This substantially flat device confines the reacting material to a structure having its longest dimension in contact with the external surroundings. That is, the confinement of the reacting material within a substantially flat sampling region provides a large ratio of surface of the device to volume of the implanted reacting material (e.g., the FRET molecules) disposed within the sampling region. Thus, for some applications of the present invention described herein implanted electro-optical fluorescence measuring systems are provided which comprise flat sampling regions having a large ratio of interface area to volume.

Analytical techniques using fluorescent molecules as indicators have classically been used in fluorescence spectrophotometers. These instruments are designed to read fluorescence intensity and also the decay time of fluorescence. These devices are typically used in research laboratories.

A second area of fluorescence sensors known in the art includes fiber optic devices. These sensor devices allow miniaturization and remote sensing of specific analytes. The fluorescence indicator molecule is immobilized via mechanical or chemical means to (1) a first end of an optical fiber and a fiber coupler (e.g., a “Y”-shaped fiber junction) or (2) a beam splitter is attached to a second end of the fiber. Incident excitation light reaches the second end of the fiber (e.g., via a first “upper” leg of the “Y”-shaped junction) typically via a filter and a lens. This excitation light propagates via the fiber toward the fluorescence indicator molecule that is immobilized at the first end. Upon excitation, the indicator molecule uniformly radiates the fluorescent light, some of which is recaptured by the first end of the fiber optic and propagates back through the fiber to the bottom leg of the “Y”-shaped fiber junction, or “coupler” at the second end of the fiber optic. At the junction, a substantial portion (e.g., at least a half) of the fluorescent light is redirected towards a second “upper” leg of the “Y”-shaped junction that guides the fluorescent light towards a photo-detector that is disposed in optical communication with the second “upper” leg of the “Y”-shaped fiber junction. A primary disadvantage with the system is the losses occurring at each fiber junction and via lenses and filters. The system is at a maximum 1-5% efficient with resultant loss in sensitivity and range. These fiber optic devices have been demonstrated in the lab and are available commercially for limited applications. These fiber optic devices differ from the previously mentioned fluorescence spectrophotometers in that they are dedicated to their specific application, because they are not devised for easy exchange of filters or of fluorescent reacting material. To offset the inefficiencies of the fiber optic system, lasers are often used to raise the input power and highly sensitive photomultiplier tubes are used as detectors (thereby raising the cost of the device by thousands of dollars).

The aforementioned prior art fluorescence spectrophotometers and fiber optic devices and technologies are not conducive to implantable devices having substantially flat sampling regions with large a interface to the surroundings. Moreover, these prior art fluorescence devices are complex and bulky with many separate parts, on top of being very expensive.

U.S. Pat. Nos. 6,671,527 and 7,228,159 to Petersson et al., describe the optics of a fiber optic miniaturized fluorimeter which comprises an external unit of a sensor for in vivo measurement of an analyte. The sensor further comprises fluorescent particles that are implanted into the body of a mammal and that are both excited and detected by the externally-disposed fluorimeter. The fluorimeter comprises a light emitting diode (LED) providing an excitation light beam which passes through a condenser containing an excitation filer and reaches a beam splitter. Part of the excitatory beam is thereby deflected into launching optics and enters an optical fiber. When the fluorimeter is in use in the interrogation of a cutaneously located sensor that is disposed adjacently to the skin, the fluorimeter is placed in alignment with the cutaneously-disposed sensor, so that the beam of excitatory light is incident upon the sensor. A portion of the optical signal emitted from the sensor following excitation enters the optical fiber and is thereby conveyed into the fluorimeter where it passes through a blocking diode. The fluorimeter also contains a reference detector diode, which provides a reference measurement of the excitatory light emitted from the LED. One end of the fiber is mounted in an X Y Z holder in front of a ×20 microscope objective

The disclosure provided in the Petersson patents is different from the applications of the invention that are disclosed herein because the Petersson patents describe a device in which there is physical separation between (1) the sampling region (i.e., the cutaneously-disposed biological sensor) that is disposed within the skin of the body of the subject, and (2) the optical monitoring system (i.e., the fluorimeter) that lies externally to the body of the subject, i.e., on the surface of the skin. Accordingly, the device described in the Petersson patents is not suitable for full implantation, in contrast with the device described herein which is designated for complete implantation (i.e., the sampling region and the optical detector are both implanted in the body of the subject). Additionally, the device described in the Petersson patents applies fiber optics which, as described hereinabove, causes significant excitation energy loss and fluorescent emission signal loss, and the device supports only a single excitation and a single detection wavelength bands, respectively.

U.S. Pat. Nos. 5,894,351, 5,910,661, 5,917,605, 6,304,766, 6,330,464, 6,711,423, 6,940,590, 7,016,714, 7,135,342, 7,157,723, 7,190,445, 7,227,156, 7,308,292, 7,375,347, and 7,405,387 to Colvin, Jr., et al., describe compact and implantable fluorescence measurement devices, all having the basic structure of smooth and rounded oblong, oval, or elliptical shape (e.g., a bean- or pharmaceutical capsule-shape). The fluorescent material is layered on the surface of the device. Light source, filters and light detectors, with all required electronics are compactly packed inside, and light conducting material fills the space in between them and the fluorescent material. In the devices described in the Colvin Jr. patents, a light source, preferably a light-emitting diode (LED), is located at least partially within the indicator material, such that incident light from the light source causes the indicator molecules to fluoresce. A long-pass filter allows emitted light from the indicator molecules to reach the light detector, while it filters out scattered incident light from the light source. An analyte is allowed to permeate through the fluorescent matrix and thereby changes the fluorescent properties of the indicator material proportionately to the amount of analyte present. The emitted fluorescent light is then detected and measured by a light detector, thus providing a measurement of the amount or concentration of analyte present within the environment of interest.

In IEEE Journal of Solid-state Circuits, vol. 41, no. 11, p. 2521 (2006), Patounakis et al. describe a CMOS biosensor substrate for fluorescence-based assays that enables time-gated, time-resolved fluorescence detection. The electronics described therein allow miniaturization of the device together with high sensitivity. In some applications of the present invention, devices described herein apply similar electronics to steady-state fluorescence, thus expanding the applicability of the technology to a larger group of longer-lasting indicator materials. Such indicator, e.g., proteins that are produced by living cells, may serve in the future in long-lasting implanted sensors. For example, these cells are produced in implantable continuous glucose monitors as described, for example in PCT Publication WO 07/110,867 to Gross et al., which is incorporated herein by reference.

The devices described herein, and respective applications of the present invention, overcome some or all of the following challenges presented by the devices described in at least some of the aforementioned references:

1. Sharing of optical paths of illumination and detection, thereby giving rise to a certain penetration of illumination light through detection filters thus distorting the accuracy of the fluorescence readings;

2. A very small size ratio between the illumination source and the fluorescent matrix to be covered by emitted light, contributing to low excitation intensity;

3. Low efficiency of light collection;

4. Large incidence angles of light reaching the long-pass fluorescence emission filters, giving rise to the transmission of unwanted light into the light detectors, thereby distorting measurements and analysis; and

5. Complicated tasks of fixing and coupling of the fluorescent material to the surface of the light-guiding material while allowing free diffusion of analyte to the fluorescent material.

Some applications of the present invention described herein overcome the aforementioned challenges and describe alternative novel devices which increase illumination power and improve light collection and detection efficiency. The devices described herein, and various applications thereof, provide (1) more freedom in the housing for mobilization of the fluorescent material, and (2) larger area of interface to the surroundings, which is achieved by providing devices having substantially flat sampling regions. Moreover, in some applications of the present invention, the flow of liquid through the sampling region area is improved, due to improved physical conditions provided by the device for material exchange between the sampling region and the surrounding body tissue and/or fluid.

There is therefore provided, in accordance with some applications of the present invention, apparatus, including:

a support configured to be implanted within a body of a subject;

a sampling region coupled to the support, the apparatus configured to passively allow passage through the sampling region of at least a portion of fluid from the subject; and

an optical measuring device in optical communication with the sampling region, including:
at least one light source configured to transmit light through at least a portion of the fluid, and
at least one sensor configured to measure a parameter of the fluid by detecting light passing through the fluid.

In some applications of the present invention, a ratio of (a) a volume of the sampling region expressed in cubic millimeters to (b) a surface area of the sampling region expressed in square millimeters, is between 1 and 14 mm.

In some applications of the present invention, the ratio of (a) the volume of the sampling region expressed in cubic millimeters to (b) the surface area of the sampling region expressed in square millimeters, is between 2 and 8 mm

In some applications of the present invention, the sampling region is shaped so as to provide two large exposed surfaces of configured for exchange of material with an area surrounding the device.

In some applications of the present invention, the portion of the fluid includes glucose, and the apparatus is configured to passively allow passage of the glucose through the sampling region.

In some applications of the present invention, the parameter of the fluid includes glucose concentration, and the optical measuring device is configured to measure a concentration of glucose in the fluid.

In some applications of the present invention, the apparatus is configured for subcutaneous implantation within the subject.

In some applications of the present invention, the fluid includes components of interstitial fluid of the subject, and the apparatus is configured to facilitate a measurement of a parameter of the interstitial fluid of the subject.

In some applications of the present invention, the light source includes one or more light sources selected from the group consisting of: a light emitting diode (LED), an organic light emitting diode (OLED), a laser diode, and a solid-state laser.

In some applications of the present invention, the light source is configured to emit visible light.

In some applications of the present invention, the light source is configured to emit infrared light.

In some applications of the present invention, the application further includes a drug administration unit configured to administer a drug in response to the measured parameter.

In some applications of the present invention, the optical measuring device includes an absorbance spectrometer.

In some applications of the present invention, the application further includes a housing coupled to the support and surrounding the sampling region, the housing having at least one opening formed therein configured for passage of the fluid therethrough and into the housing.

In some applications of the present invention, the apparatus further includes a transmitter and a receiver, the transmitter configured to be in communication with the sensor, and the receiver configured to be disposed at a site outside the body of the subject, and the transmitter is configured to transmit the measured parameter to the receiver.

In some applications of the present invention:

the support is shaped to define a cylindrical support defining a lumen thereof, and

the sampling region is disposed within the lumen.

In some applications of the present invention, the apparatus further includes cells disposed within the sampling region, the cells being genetically engineered to produce, in situ, a protein configured to facilitate a measurement of the parameter of the fluid.

In some applications of the present invention, the light source includes a plurality of light sources, and the sensor includes a plurality of photodetectors.

In some applications of the present invention, the light source is configured to emit polarized light, and the apparatus further includes at least one first polarizing filter having an orientation thereof and configured to filter the polarized light emitted from the light source into the sampling region.

In some applications of the present invention:

the support is shaped to define a wall thereof surrounding the sampling region,

the at least one light source includes a plurality of light sources disposed along the wall of the support and configured to transmit light through the sampling region, and

the at least one sensor includes a plurality of sensors disposed along the wall of the support and configured to receive at least a portion of the light passing through the fluid.

In some applications of the present invention, the light source and the sampling region are disposed at a first horizontal plane of the device, and the at least one sensor is disposed at a second horizontal plane of the device.

In some applications of the present invention, the light source is configured to transmit fluorescence exciting light to the sampling region from a direction that is at a non-zero angle with respect to a direction of a central axis of a light beam that originates in the sampling region and propagates toward the at least one sensor.

In some applications of the present invention, the light source is configured to transmit fluorescence exciting light to the sampling region from a direction that is substantially perpendicular to a direction of a central axis of a light beam that originates in the sampling region and propagates toward the at least one sensor.

In some applications of the present invention, the light source and the sampling region are disposed at a first horizontal plane of the device, and the at least one sensor is disposed at a second horizontal plane of the device.

In some applications of the present invention, the sampling region includes a permeable material selected from the group consisting of: agarose, silicone, polyethylene glycol, gelatin, an optical fiber capillary, a polymer, a co-polymer, an extracellular matrix, and alginate, the permeable material being positioned to passively allow passage therethrough of the portion of fluid in the sampling region.

In some applications of the present invention, the material includes an optically-transparent and glucose-permeable material.

In some applications of the present invention, the material is configured to restrict passage of cells into and out of the sampling region.

In some applications of the present invention, the apparatus further includes at least one selectively-permeable membrane coupled to the support.

In some applications of the present invention, the membrane is configured to restrict passage of cells into and out of the sampling region.

In some applications of the present invention, the support has a first surface and a second surface, and the at least one selectively-permeable membrane includes:

a first selectively-permeable membrane coupled to the first surface; and

a second selectively permeable membrane coupled to the second surface.

In some applications of the present invention:

the fluid includes components of blood of the subject,

the support is configured for implantation within a blood vessel of the subject, and

the apparatus is configured to facilitate a measurement of a parameter of blood of the subject.

In some applications of the present invention, the blood vessel includes a vena cava of the subject, and the support is configured for implantation within the vena cava of the subject.

In some applications of the present invention, the optical measuring device is configured to be disposed externally to the blood vessel, and the optical measuring device is configured to be in optical communication with a vicinity of the blood vessel in which the support is implanted.

In some applications of the present invention, the support is shaped to define a cylindrical support, the cylindrical support defining a lumen thereof that surrounds the sampling region.

In some applications of the present invention, the apparatus further includes at least one optical fiber; the optical fiber is coupled at a first end to the optical measuring device, and at a second end to the support, and light from the light source is provided to the sampling region via the optical fiber.

In some applications of the present invention, the parameter of the blood includes a level of glucose in the blood, and the apparatus is configured to facilitate a measurement of the level of glucose in the blood of the subject.

In some applications of the present invention, the apparatus further includes a tunable filter configured to refract the light emitted from the light source into a plurality of monochromatic bands.

In some applications of the present invention, the tunable filter includes a Faraday rotator.

In some applications of the present invention, the sensor includes a plurality of photodetectors, each photodetector detecting a respective one of the plurality of monochromatic bands.

In some applications of the present invention, the apparatus further includes at least one reflector, configured to reflect to the sensor light emitted from the light source that has passed through the sampling region.

In some applications of the present invention, the at least one reflector includes a plurality of reflectors, each one of the plurality of reflectors is disposed at a respective location with respect to the sampling region, and the plurality of reflectors lengthens an optical path between the light source and the sensor.

In some applications of the present invention, the sampling region has at least one surface thereof configured for the passage of the portion of fluid therethrough, the surface having a surface area that is at least 50% of a total surface area of the apparatus.

In some applications of the present invention, the sampling region has at least one surface thereof configured for the passage of the portion of fluid therethrough, the surface having a surface area that is at least 70% of a total surface area of the apparatus.

In some applications of the present invention, the sampling region has a length between 1 mm and 10 mm.

In some applications of the present invention, the sampling region has a length between 10 mm and 100 mm.

In some applications of the present invention, the sensor is configured to measure the light scattered within the sampling region.

In some applications of the present invention, the light source and the sensor are physically separated by at least a portion of the sampling region.

In some applications of the present invention, the support is configured for implantation within a blood vessel of the subject, and the optical measuring device is configured to be in optical communication with a vicinity of the blood vessel in which the support is implanted.

In some applications of the present invention, the blood vessel includes a vena cava of the subject, and the support is configured for implantation within the vena cava of the subject.

In some applications of the present invention, the support is shaped to define a cylindrical support, and the sampling region is disposed within the wall of the cylindrical support.

In some applications of the present invention, the support includes a disc-shaped support.

In some applications of the present invention, the support is shaped to define a cylindrical support, the cylindrical support defining a lumen thereof that surrounds the sampling region.

In some applications of the present invention, the apparatus further includes cells disposed within the sampling region, the cells being genetically engineered to produce, in situ, a protein configured to facilitate a measurement of the parameter of the blood.

In some applications of the present invention:

the support is shaped to define a cylindrical support defining a lumen thereof,

the sampling region is disposed within the lumen, and

the cells are genetically engineered to secrete the protein into the sampling region.

In some applications of the present invention, the optical measuring device is configured to be disposed externally to the blood vessel.

In some applications of the present invention, the apparatus further includes at least one optical fiber, the optical fiber is coupled at a first end to the optical measuring device, and at a second end to the support, and light from the light source is provided to the sampling region via the optical fiber.

In some applications of the present invention, the fluid includes components of blood of the subject, and the apparatus is configured to facilitate a measurement of a parameter of the blood of the subject.

In some applications of the present invention, the parameter of the blood includes a level of glucose in the blood, and the apparatus is configured to facilitate a measurement of the level of glucose in the blood of the subject.

In some applications of the present invention, the sensor is configured to measure the parameter by detecting a photoacoustic effect induced by the light passing through the fluid.

In some applications of the present invention, the light source includes a solid-state laser.

In some applications of the present invention, the light source is configured to emit visible light.

In some applications of the present invention, the sensor includes a photodetector.

In some applications of the present invention, the light source includes a plurality of light sources, and the sensor includes a plurality of photodetectors.

In some applications of the present invention, the light source is configured to emit polarized light, and the apparatus further includes at least one first polarizing filter having an orientation thereof and configured to filter the polarized light emitted from the light source into the sampling region.

In some applications of the present invention, the application further includes at least one second polarizing filter configured to filter to the sensor the polarized light passing through the sampling region.

In some applications of the present invention, the second polarizing filter has an orientation thereof that is substantially perpendicular to the orientation of the first polarizing filter.

In some applications of the present invention, the light includes visible light, and the apparatus further includes a tunable filter configured to refract the light emitted from the light source into a plurality of monochromatic bands.

In some applications of the present invention, the tunable filter includes a Faraday rotator.

In some applications of the present invention, the sensor includes a plurality of photodetectors, each photodetector detecting a respective one of the plurality of monochromatic bands.

In some applications of the present invention, the sampling region includes a gel including extracellular matrix and a permeable material selected from the group consisting of: agarose, silicone, polyethylene glycol, gelatin, an optical fiber capillary, a polymer, a co-polymer, and an alginate.

In some applications of the present invention, the gel includes an optically-transparent and glucose-permeable gel.

In some applications of the present invention, the gel is configured to restrict passage of cells into the sampling region.

In some applications of the present invention, the apparatus further includes a selectively-permeable membrane coupled to the support, the membrane being configured to surround the sampling region.

In some applications of the present invention, the fluid includes interstitial fluid, and the membrane is configured to restrict passage therethrough of cells.

In some applications of the present invention, the support includes a disc-shaped housing, and the sampling region includes a disc-shaped sampling region.

In some applications of the present invention, the sampling region has at least one surface thereof configured for the passage of the portion of fluid therethrough, the surface having a surface area that is at least 50% of a total surface area of the apparatus.

In some applications of the present invention, the sampling region has at least one surface thereof configured for the passage of the portion of fluid therethrough, the surface having a surface area that is at least 70% of a total surface area of the apparatus.

In some applications of the present invention:

the support is shaped to define a wall thereof surrounding the sampling region,

the at least one light source includes a plurality of light sources disposed along the wall of the support and configured to transmit light through the sampling region, and

the at least one sensor includes a plurality of sensors disposed along the wall of the support and configured to receive at least a portion of the light passing through the fluid.

In some applications of the present invention, the support has a first surface and a second surface, and the apparatus further includes a first selectively-permeable membrane coupled to the first surface and a second selectively permeable membrane coupled to the second surface.

In some applications of the present invention, the first and second selectively-permeable membranes are configured to restrict passage of cells therethrough.

There is additionally provided, in accordance with some applications of the present invention, apparatus, including:

a support configured to be implanted within a body of a subject;

at least one membrane coupled to the support configured to define a sampling region, the membrane configured to passively allow passage through the sampling region of a fluid from the subject; and

an optical measuring device in optical communication with the sampling region, including:
at least one light source configured to transmit light through at least a portion of the fluid, and
at least one sensor configured to measure a parameter of the fluid by detecting light that has passed through the fluid.

In some applications of the present invention, the membrane has at least one surface thereof configured for the passage of the portion of fluid therethrough, the surface having a surface area that is at least 50% of a total surface area of the apparatus.

In some applications of the present invention, the membrane has at least one surface thereof configured for the passage of the portion of fluid therethrough, the surface having a surface area that is at least 70% of a total surface area of the apparatus.

In some applications of the present invention, the membrane is configured to restrict passage of cells therethrough.

In some applications of the present invention, the sampling region includes a permeable material selected from the group consisting of: silicone, a polymer, and an alginate, the permeable material being positioned to passively allow passage therethrough of the fluid in the sampling region.

In some applications of the present invention, the support includes a disc-shaped housing, and the sampling region includes a disc-shaped sampling region.

In some applications of the present invention, the sampling region has at least one surface thereof configured for the passage of the portion of fluid therethrough, the surface having a surface area that is at least 50% of a total surface area of the apparatus.

In some applications of the present invention, the sampling region has at least one surface thereof configured for the passage of the portion of fluid therethrough, the surface having a surface area that is at least 70% of a total surface area of the apparatus.

In some applications of the present invention:

the support is shaped to define an annular wall thereof surrounding the sampling region,

the at least one light source includes a plurality of light sources disposed along the wall of the support and configured to transmit light through the sampling region, and

the at least one sensor includes a plurality of sensors disposed along the wall of the support and configured to receive at least a portion of the light passing through the fluid.

In some applications of the present invention, the support has a first surface and a second surface, and the at least one membrane includes a first selectively-permeable membrane coupled to the first surface and a second selectively-permeable membrane coupled to the second surface.

In some applications of the present invention, the first and second selectively-permeable membranes are configured to restrict passage of cells therethrough.

There is further provided, in accordance with some applications of the present invention, an optical sensor device for determining the intensity of fluorescent emitted light, including:

a sampling region having at least one side thereof configured for exchange of material with a vicinity surrounding the device, the sampling region having a ratio of (a) a volume of the sampling region expressed in cubic millimeters to (b) a surface area of the sampling region expressed in square millimeters, is between 1 and 14 mm;

at least one light source that produces fluorescence excitation light, the light source being in optical communication with the sampling region;

at least one filter in optical communication with the sampling region and configured to filter fluorescence emission wavelength bands of light from the sampling region, in response to light transmitted from the light source; and

at least one light detector configured to detect light emitted from the sampling region that has passed through the at least one filter.

In some applications of the present invention, the light source includes one or more light sources selected from the group consisting of: a light emitting diode (LED), an organic light emitting diode (OLED), a laser diode, surface-emitting laser, and a solid-state laser.

In some applications of the present invention, the light source includes two or more light source units.

In some applications of the present invention, the each one of the two or more light source units emits light having two or more wavelength bands.

In some applications of the present invention:

the sampling region includes fluorescent material configured to emit light responsively to light transmitted from the light source,

the device further includes one or more component selected from the group consisting of: at least one filter and at least one optical element, disposed between the light source and the sampling region, and

the selected component is configured for selecting light having at least one wavelength band appropriate for fluorescence excitation of the fluorescent material disposed within the sampling region.

In some applications of the present invention, the selected component selects light having two or more wavelength bands.

In some applications of the present invention, the light detector includes a photo-diode.

In some applications of the present invention, the at least one filter filters light of two or more wavelength bands.

In some applications of the present invention, the sampling region includes fluorescent material configured to emit light responsively to light transmitted from the light source, the at least one filter and the at least one light detector are arranged in a way that minimizes the effect of non-homogeneous distribution of the fluorescent material within the sampling region.

In some applications of the present invention, the device further includes one or more optical elements disposed between the light source and the sampling region and configured to guide the light from the light source into the sampling region.

In some applications of the present invention, the one or more optical elements is selected from the group consisting of: at least one light guide and at least one lens.

In some applications of the present invention, the light source is configured to transmit fluorescence exciting light to the sampling region from a direction that is at a non-zero angle with respect to a direction of a central axis of a light beam that originates in the sampling region and propagates toward the at least one light detector.

In some applications of the present invention, the light source is configured to transmit fluorescence exciting light to the sampling region from a direction that is substantially perpendicular to a direction of a central axis of a light beam that originates in the sampling region and propagates toward the at least one light detector.

In some applications of the present invention, the light source and the sampling region are disposed at a first horizontal plane of the device, and the at least one detector is disposed at a second horizontal plane of the device.

In some applications of the present invention, the device further includes one or more optical elements between the sampling region and the detector, the one or more optical elements being configured to focus light emitted from the sampling region toward the light detector.

In some applications of the present invention, the one or more optical elements includes one or more lenses.

In some applications of the present invention, the device further includes one or more folding optical elements configured to reduce at least one physical dimension of the device, the one or more folding optical element is selected from the group consisting of: mirrors, rhomboid-shaped elements, prism-shaped elements, and beam splitters.

In some applications of the present invention, the device further includes at least a first beam splitter disposed between the sampling region and the detector, the beam splitter being configured to split a fluorescence emission light beam from the sampling region into first and second beams having respective wavelength bands.

In some applications of the present invention, the device further includes at least a second beam splitter disposed between the first beam splitter and the detector, the second beam splitter being configured to:

direct at least one of the first and second beams away from the second splitter, and

filter at least the other one of the first and second beams.

In some applications of the present invention, the device further includes reflective optical elements coupled to at least a portion of the sampling region.

In some applications of the present invention, the reflective optical elements are selected from the group consisting of: mirrors and dichroic filters.

In some applications of the present invention, the device further includes optically-transparent material disposed within the device in spaces between components of the device.

In some applications of the present invention, the optically-transparent material includes a low-refractive index polymer.

In some applications of the present invention, the optically-transparent material includes a polymer selected from the group consisting of: epoxy, silicone and parylene.

In some applications of the present invention, the sampling region is shaped so as to provide two large surfaces of configured for exchange of material with an area surrounding the device.

In some applications of the present invention:

the sampling region is shaped so as to define one or more exposed large surface and one or more exposed narrow width-sides disposed perpendicularly to the one or more exposed large surfaces,

the device further includes one or more optical elements disposed between the sampling region and the detector, and

the one or more optical elements is configured to collect fluorescent emitted light from the sampling region and direct the collected light toward the light detector.

In some applications of the present invention, the one or more optical elements is selected from the group consisting of: light guides and lenses.

In some applications of the present invention, the one or more optical elements includes one or more beam expanding light guides, and successive cross-sectional lengths expand from the narrow width-side of the sampling region to the one or more optical elements.

In some applications of the present invention, at least a part of the one or more large exposed surfaces of the sampling region is covered by the one or more optical elements.

In some applications of the present invention, the sampling region is shaped so as to define one or more exposed large surface and one or more exposed narrow width-sides disposed perpendicularly to the one or more exposed large surfaces, and the device further includes one or more transmitting optical elements configured to guide light from the light source toward the one or more large surfaces of the sampling region.

In some applications of the present invention, the one or more transmitting optical elements includes one or more elements selected from the group consisting of: a cylindrical reflector and a conical reflector.

In some applications of the present invention, the one or more transmitting optical elements includes one or more light guides.

In some applications of the present invention, the device further includes one or more collecting optical elements disposed between the sampling region and the detector, the one or more collecting optical elements being configured to collect fluorescent emitted light from at least the one or more large surfaces of the sampling region and to transmit the collected light toward the light detector.

In some applications of the present invention:

the device further includes one or more filters disposed between the sampling region and the detector,

the one or more collecting optical elements includes a light guide that is disposed at a distance from the sampling region in a manner which selects a light-angular content that is optimal for the appropriate performance of the one or more filters disposed between the sampling region and the detector, and

the one or more filters select fluorescence emission wavelength bands of light from the sampling region.

In some applications of the present invention, the light guide is externally coated at least in part by reflective material, and the reflective material is configured to prevent ambient light from entering the one or more collecting optical elements.

There is also provided, in accordance with some applications of the present invention, an optical sensor device for determining the intensity of fluorescent emitted light, including:

a light-detector array;

at least a first array of filters adjacent to the detector array to select fluorescent emission wavelength bands transmitted toward the light-detector array;

a substantially flat sampling region disposed adjacent to the array of filters, the sampling region having at least one side thereof configured for exchange of material with a vicinity surrounding the device, the sampling region having a ratio of (a) a volume of the sampling region expressed in cubic millimeters to (b) a surface area of the sampling region expressed in square millimeters, is between 1 and 14 mm; and

at least one light source that produces fluorescence excitation light.

In some applications of the present invention, the light detector array defines a first light detector array, and the device further includes a second array of filters disposed between the light source and the sampling region, the second array of filters being configured to select fluorescence excitation wavelength bands of light are transmitted from the light source.

In some applications of the present invention, the two or more light source units are configured to emit toward the sampling region light having two or more wavelength bands.

In some applications of the present invention, the second array of filters is configured to filter therethrough two or more wavelength bands from the two or more light sources, a first portion of the second filter array is configured to filter light having a first one of the two or more wavelength bands, and a second portion of the second filter array is configured to filter light having a second one of the two or more wavelength bands.

In some applications of the present invention, the light-detector array includes detectors that are selected from a group consisting of: complementary metal oxide semiconductor (CMOS), charged coupled device (CCD), electron multiplying CCD (EMCCD), intensified CCD (ICCD), and electron bombardment CCD (EBCCD).

In some applications of the present invention, the first array of filters is configured to filter toward the light-detector array light in two or more wavelength bands.

In some applications of the present invention, the first array of filters is arranged in a manner which minimizes an effect of non-homogeneous distribution of fluorescent material within the sampling region.

In some applications of the present invention, the device further includes one or more optical elements disposed between the sampling region and the light-detector array, the one or more optical elements being configured to collect light from the sampling region and transmit the collected light toward the light-detector array.

In some applications of the present invention, the one or more optical elements includes a micro-lens array.

In some applications of the present invention, the device is shaped so as to define an array of pinholes between the at least one light source, the array of pinholes being configured to limit angular content of light transmitted from the at least one light source.

In some applications of the present invention, the device further includes optically-transparent material disposed within the device in spaces between components of the device.

In some applications of the present invention, the optically-transparent material includes a low-refractive index polymer.

In some applications of the present invention, the optically-transparent material includes a polymer selected from the group consisting of: epoxy, silicone and parylene.

There is also provided, in accordance with some applications of the present invention, the following inventive concepts:

1. A method for detecting a parameter of a fluid, comprising:

implanting within a body of a subject a support coupled to a sampling region configured to passively allow passage therethrough of at least a portion of the fluid;

restricting passage of cells into the sampling region;

transmitting light through the portion of the fluid; and

in conjunction with the transmitting, measuring the parameter of the fluid by detecting passage of light through the fluid.

2. The method according to inventive concept 1, wherein the portion of the fluid includes glucose, and wherein measuring the parameter comprises measuring a level of the glucose in the fluid.
3. The method according to inventive concept 1, wherein implanting comprises subcutaneously implanting.
4. The method according to inventive concept 1, wherein detecting the passage of light through the fluid comprises detecting scattering of the light passing through the fluid.
5. The method according to inventive concept 1, wherein measuring the parameter in the fluid comprises reflecting toward a sensor the light passing through the fluid.
6. The method according to inventive concept 1, further comprising administering a drug in response to the measuring.
7. The method according to inventive concept 1, further comprising implanting within the sampling region cells that are genetically engineered to express a protein configured to measure the parameter of the fluid.
8. The method according to inventive concept 1, wherein implanting comprises implanting within the body of the subject a disc-shaped housing including a disc-shaped sampling region, the disc-shaped sampling region having at least one surface thereof configured for the passage of the portion of fluid therethrough, the surface having a surface area that is at least 50% of a total surface area of the housing.
9. The method according to inventive concept 1, wherein implanting comprises implanting within the body of the subject a disc-shaped housing including a disc-shaped sampling region, the disc-shaped sampling region having at least one surface thereof configured for the passage of the portion of fluid therethrough, the surface having a surface area that is at least 70% of a total surface area of the housing.
10. The method according to inventive concept 1, wherein measuring the parameter comprises measuring the parameter using polarimetry.
11. The method according to inventive concept 10, wherein transmitting comprises transmitting polarized light through the portion of the fluid.
12. The method according to inventive concept 1, wherein implanting the support in the body of the subject comprises implanting the support in a blood vessel of the subject, and wherein measuring the parameter of the fluid comprises measuring a parameter of blood of the subject.
13. The method according to inventive concept 12, wherein implanting the support in the blood vessel of the subject comprises implanting the support in a vena cava of the subject.
14. The method according to inventive concept 12, wherein transmitting light through the portion of the fluid comprises transmitting light through the blood of the patient.

The present invention will be more fully understood from the following detailed description of some applications thereof; taken together with the drawings, in which:

### BRIEF DESCRIPTION OF THE DRAWINGS

FIG. 1 is a schematic illustration of an optical measuring device, in accordance with some applications of the present invention;

FIGS. 2 and 3 are schematic illustrations of the optical measuring device of FIG. 1, in accordance with respective applications of the present invention;

FIG. 4 is a schematic illustration of an optical measuring device, in accordance with some other applications of the present invention;

FIG. 5 is a schematic illustration of the optical measuring device of FIG. 4, in accordance with some applications of the present invention;

FIG. 6 is a schematic illustration of the optical measuring device comprising genetically-engineered cells, in accordance with some applications of the present invention;

FIG. 7 is a schematic illustration of the optical measuring device, in accordance with still some other applications of the present invention;

FIG. 8 is a schematic illustration of a disc-shaped optical measuring device, in accordance with some applications of the present invention;

FIG. 9 is a schematic illustration of a sampling region disposed within a blood vessel of the subject, in accordance with some applications of the present invention;

FIG. 10 is a schematic illustration of an optical sensor device, in accordance with some applications of the present invention;

FIG. 11A-C is a schematic illustration of the optical sensor device, in accordance with some other applications of the present invention;

FIG. 12 is a schematic illustration of an optical sensor device comprising a beam expander and a dichroic filter, in accordance with some applications of the present invention;

FIG. 13 is a schematic illustration of an optical sensor device comprising two analyte sampling regions, in accordance with some applications of the present invention;

FIG. 14 is a schematic illustration of an optical sensor device comprising prisms, in accordance with some applications of the present invention;

FIG. 15 is a schematic illustration of an optical sensor device comprising a beam splitter and a folding mirror, in accordance with some applications of the present invention;

FIG. 16 is a schematic illustration of an optical sensor device comprising a reflective cylinder and cone, in accordance with some applications of the present invention;

FIGS. 17-18 are schematic illustrations of an optical sensor device comprising reflective conical surfaces, in accordance with some other applications of the present invention;

FIG. 19 is a schematic illustration of an optical sensor device comprising rhomboid optical light guides; in accordance with some applications of the present invention; and

FIGS. 20-22 are schematic illustration of an optical sensor device comprising an array of detectors disposed in a plane in parallel with an array of filters, in accordance with some applications of the present invention.

### DETAILED DESCRIPTION OF THE EMBODIMENTS

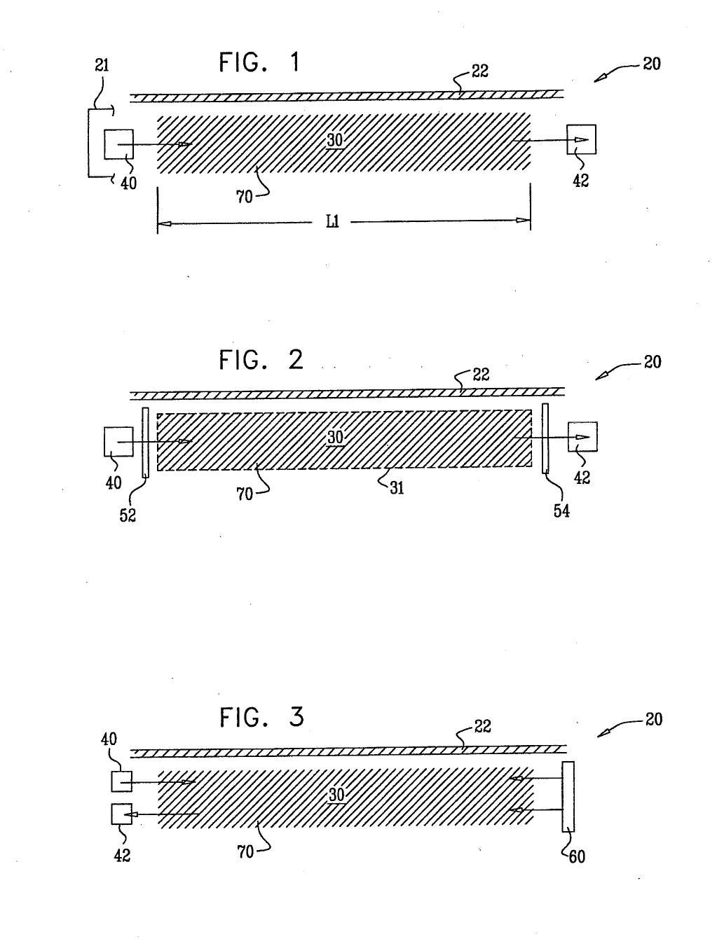

Reference is now made to FIG. 1, which is a schematic illustration of an optical measuring device 20 comprising an electromagnetic light source 40 and a detecting system 42, in accordance with some applications of the present invention. Typically, light source 40 is configured to emit electromagnetic radiation that is in the visible or infrared range. Optical measuring device 20 is configured to detect and measure a concentration of an analyte, e.g., glucose, in interstitial fluid of a subject. (In the context of the specification, examples of the analyte being glucose are by way of illustration and not limitation.) Typically, device 20 is designated for subcutaneous implantation under skin 22 of a subject and comprises a support 21 (e.g., a housing, a scaffold, or glue). A sampling region 30 is disposed within an area defined by support 21 of device 20, typically between light source 40 and detecting system 42 (as shown). Support 21 is configured to facilitate proper spatial relationship between sampling region 30, source 40, and detecting system 42.

In some applications of the present invention, light source 40 comprises any suitable light source, e.g., a light emitting diode (LED), an organic light emitting diode (OLED), a laser diode, or a solid-state laser.

In some applications of the present invention, support 21 comprises a selectively-permeable membrane, and support 21 is coupled to the selectively-permeable membrane. In some applications of the present invention, the membrane is optically transparent. Typically, the membrane is permeable to molecules having a molecular weight equal to or less than the molecular weight of the analyte (e.g., glucose) configured to be measured by device 20. Typically, the membrane is configured to restrict passage into sampling region 30 of cells from outside device 20.

Sampling region 30 comprises an optically-transparent and glucose-permeable material 70. In some applications of the present invention, material 70 comprises, by way of illustration and not limitation, alginate, agarose, silicone, a polymer, a co-polymer polyethylene glycol (PEG), and/or gelatin. Alternatively or additionally, material 70 comprises a glucose-permeable gel comprising extracellular matrix (ECM) in combination with one or more of the above-listed optically-transparent and glucose-permeable materials. For some applications of the present invention, material 70 comprises an optically-transparent and glucose-permeable copolymer, e.g., Poly (dimethyl siloxane) (PDMS), Poly (N-isopropyl acrylamide) (PNIPAAM), and other optically-transparent and glucose-permeable copolymers, or other copolymers known in the art. In some applications of the present invention, material 70 comprises a plurality of hollow capillary fibers configured for optical transmission of the light from source 40 and to allow for passage of certain constituents (e.g., small molecules such as glucose) of fluid through sampling region 30 in order to facilitate optical measuring of the analyte in region 30.

Typically, material 70 is configured to passively allow passage therethrough of certain constituents (e.g., small molecules such as glucose) of the interstitial fluid of the subject that have a molecular weight smaller than the desired molecular weight cutoff defined by material 70. For example, the molecular weight cutoff allows passage therethrough of glucose molecules present in the interstitial fluid. In some applications of the present invention, the molecular weight cutoff allows passage through material 70 of only glucose molecules present in the interstitial fluid and of other molecules having a molecular weight equal to or less than the molecular weight of the glucose molecule. That is, material 70 is configured to restrict passage therethrough into sampling region 30 of molecules having a molecular weight greater than a molecular weight of a glucose molecule.

Material 70 defining sampling region 30 has suitable fixed dimensions such that the glucose concentration within region 30 is in equilibrium with glucose concentrations of adjacent interstitial fluid not within region 30. The fixed dimensions of region 30 enable passage therethrough of a definite, consistent, relatively small volume, e.g., up to 1 mL, of fluid during each measurement. In some applications of the present invention, sampling region 30 enables passage therethrough of a volume between 0.01 mL and 1 mL, e.g., between 0.05 mL and 0.5 mL, of fluid during each measurement. Sampling region 30 has a length L1 of between 1 mm and 100 mm.

Because of the typically small size of region 30, the concentration of the analyte outside of device 20 is in general equilibrium with the average concentration of analyte in the fluid measured in region 30 during measurements thereof. Therefore, measuring the concentration of glucose in sampling region 30 provides an indication of the concentration of glucose in the body of the subject.

Typically, material 70 has a relative refractive index of about 1.35-1.40, which prevents or minimizes loss of light and refraction thereof.

Material 70 permits passage therethrough of the constituents of the interstitial fluid having a molecular weight smaller than the molecular weight cutoff defined by material 70. Typically, material 70 restricts passage of cells from outside device 20 and into sampling region 30. This permeability typically does not affect the equilibrium of glucose concentrations between the fluid inside sampling region 30 and the interstitial fluid not inside device 20.

Typically, light source 40 transmits light through sampling region 30 and toward detecting system 42 in the direction indicated by the arrows in the figure. For some applications of the present invention in which source 40 emits polarized light, the light is rotated by the glucose in sampling region 30. Detecting system 42 typically comprises a sensor (e.g., a photodetector such as a linear detector) for detecting the rotation of the light that has passed through region 30. In some applications of the present invention, detecting system 42 comprises an array of sensors.

A control unit, e.g., a microprocessor (not shown), is typically in communication with device 20 and facilitates real-time quantitative analysis of glucose in sampling region 30. Typically, the control unit drives light source 40 to emit light within region 30 in accordance with various emission parameters, such as duty cycle (e.g., number and/or timing of hours of operation per day), wavelengths, and amplitudes. In some applications of the present invention, light source 40 is actuated by the control unit using a duty cycle of less than 0.02% (e.g., on for 10 msec, off for 1 minute). Alternatively, the control unit is configured to drive light source 40 to emit light with a different duty cycle, or continuously. In some applications of the present invention, the control unit is externally programmable following implantation to allow calibration or intermittent optimization of the various emission parameters of light source 40. A power source (not shown) is coupled to device 20 and is configured to supply power thereto.

In some applications of the present invention, the control unit of device 20 is coupled to a drug administration unit (not shown) configured to administer a drug in response to the measured parameter, e.g., in response to the level of glucose measured in the interstitial fluid. In some applications of the present invention, the drug administration unit comprises an insulin pump, which supplies insulin or another drug to the body in response to the level of the analyte determined by device 20.

Reference is now made to FIG. 2, which is a schematic illustration of device 20 as described hereinabove with reference to FIG. 1, with the exception that device 20 comprises optical filters 52 and 54, in accordance with some applications of the present invention. Typically, optical measuring device 20 is configured to measure the concentration of glucose in sampling region 30 using at least one technique such as polarimetry, absorbance spectroscopy, and/or other optical measuring techniques known in the art.

It is to be noted that although device 20 is shown comprising two filters 52 and 54, device 20 may comprise one, both, or neither of filters 52 and 54. For some applications of the present invention in which filters 52 and 54 are polarizing filters, filter 54 is disposed substantially perpendicularly with respect to filter 52.

As shown, a selectively-permeable, biocompatible membrane 31 is disposed around region 30, and is configured to restrict passage of cells into sampling region 30. In some applications of the present invention, membrane 31 comprises a hydrophobic membrane, e.g., a nitrocellulose membrane. In some applications of the present invention, membrane 31 comprises a polyvinylidene difluoride, or PVDF, membrane. In some applications of the present invention, membrane 31 has a molecular weight cutoff of around 500 kDa. It is to be noted, however, that applications described herein may be implemented independently of membrane 31.

Membrane 31 provides permeability for passage therethrough of certain constituents (e.g., small molecules such as glucose) of the interstitial fluid that have a molecular weight smaller than the molecular weight cutoff defined by membrane 31. Typically, membrane 31 allows the passage into sampling region 30 of molecules having a molecular weight below a desired cut-off. For example, the molecular weight cutoff allows passage through membrane 31 of only glucose molecules present in the interstitial fluid and of other molecules having a molecular weight less than or generally equal to the molecular weight of the glucose molecule. That is, membrane 31 is configured to restrict passage therethrough into sampling region 30 of molecules or other body fluid components having a molecular or characteristic weight substantially greater than the molecular weight of a glucose molecule, e.g., cells.

This permeability typically does not affect the equilibrium of glucose concentrations between the fluid inside the device and the interstitial fluid not inside the device, as described hereinabove with respect to material 70 with reference to FIG. 1.

In some applications of the present invention, membrane 31 is optically transparent. Typically, membrane 31 is permeable to molecules having a molecular weight less than or generally equal to the molecular or characteristic weight of the analyte (e.g., typically, glucose) that is measured by device 20. Typically, membrane 31 restricts passage into sampling region 30 of cells from outside device 20.

Membrane 31 defines sampling region 30 and has suitable fixed dimensions such that the glucose concentration within region 30 is in general equilibrium with glucose concentrations of the interstitial fluid not within region 30, as described hereinabove with reference to material 70 (with reference to FIG. 1). The fixed dimensions of region 30 enable passage therethrough of a definite, consistent, relatively small volume, e.g., up to about 1 mL, of fluid during each measurement.

It is to be noted that membrane 31 is shown by way of illustration and not limitation. For example, material 70 of sampling region 30 may be disposed within an area defined by a support (as described hereinabove with reference to FIG. 1) and/or upon a scaffold independently of or in combination with membrane 31. The scaffold may comprise a porous material configured to allow passage therethrough into region 30 of constituents of the interstitial fluid having a molecular weight smaller than the desired molecular weight cutoff defined by the scaffold. Typically, the scaffold is configured to restrict passage of cells into sampling region 30. The scaffold may be used independently of or in combination with membrane 31.

In the following techniques, device 20 comprises filters 52 and 54 in order to facilitate detection of glucose concentration using polarimetry techniques known in the art. Polarimetry techniques described herein are typically practiced in combination with polarimetry techniques described in one or more of the references in the Background section of the present patent application, namely:
U.S. Pat. No. 5,209,231;
U.S. Pat. No. 6,188,477;
U.S. Pat. No. 6,577,393;
Wan Q, “Dual wavelength polarimetry for monitoring glucose in the presence of varying birefringence,” A thesis submitted to the Office of Graduate Studies of Texas A&M University (2004); and
Yu-Lung L et al., “A polarimetric glucose sensor using a liquid-crystal polarization modulator driven by a sinusoidal signal,” Optics Communications 259(1), pp. 40-48 (2006).

All of these references are incorporated herein by reference.

Typically, light emitted from light source 40 includes wavelengths within the visible range. In such an application, light source 40 comprises an incandescent light bulb, by way of illustration and not limitation, and the linear polarization vector of light rotates when the light is passed from light source 40 through region 70 comprising a chiral analyte, e.g., glucose. The rotation measured is proportional to the concentration of the glucose being monitored.

In addition to the dependence of the rotation of the linear vector of the polarized light on the concentration of the chiral analyte, the amount of the rotation of the linear vector of the polarized light also depends on (1) the properties, e.g., optical path length, defined by region 30, and (2) the wavelength of the light used for the measurement. The relationship between a degree of optical rotation and the concentration of glucose in region 30, in addition to parameters of region 30, is expressed in the following equation:

Phi=(alpha.sub.lambda)LC, (eq. 1).

where phi is the angle of rotation, alpha.sub.lambda is the specific rotation at a wavelength lambda, L is the path length, and C is the concentration of glucose in region 30. Due to the dependency of concentration measurement upon the optical path length provided by sampling region 30, material 70 is optically-transparent and glucose-permeable, and has a particular length. Additionally, the physical length of sampling region 30 can be reduced while still maintaining a desired optical path length. For example, region 30 may have a physical length that is shorter than the optical path length of the light emitted from source 40. That is, region 30 may comprise at least one mirror configured to reflect the light and thereby lengthen the optical path length. In some applications of the present invention, the mirror may be disposed externally to region 30 and in communication therewith.

In some applications of the present invention, region 30 may comprise at least one hollow optical waveguide, e.g., in the form of a light-conductive capillary fiber, in order to elongate the optical path of the light emitted from light source 40. For applications in which one waveguide is used, the waveguide may be wound or bundled in order to elongate the optical path of the light emitted from light source 40. In such an application, the analyte passes through the hollow waveguide.

Subcutaneous implantation of device 20 prevents any unwanted depolarization that is typically caused by transmitting a beam of polarized light through skin 22 of the subject, because the thickness of skin 22 typically provides a small optical path length and generates a relatively low signal-to-noise ratio. The subcutaneous positioning of device 20 in combination with the characteristics of regions 30 (described hereinabove) enables light to be passed from source 40, and through region 30, without substantial depolarization of the light.

In order to ensure that the rotated light will pass through filter 54, device 20 provides a suitable length of region 30 that is typically between 1 mm and 10 mm, or between 10 mm and 100 mm.

It is to be noted that region 30 may have any suitable optical path length in accordance with a level of specificity and sensitivity of device 20, typically, 10 mm. Increasing the optical path length provided by region 30 increases the sensitivity of device 20.

For applications of the present invention in which the level of glucose is measured using polarimetric techniques described herein, device 20 comprises a beam-splitter configured to split the beam from light source 40 into two or more beams, e.g., a principle beam and a reference beam. In some applications of the present invention, at least one reference beam, e.g., two reference beams, are created, and the reference beam(s) passes through a polarized filter. In some applications of the present invention, the principle beam and the reference beam have substantially equal optical paths that differ only in factors that support quantitative evaluation of the concentration of the analyte, e.g., glucose, in material 70 within region 30. For example: (a) sampling region 30 may provide a portion of material 70 that is configured to not contain fluid, and (b) the reference beam may be directed through the portion of region 30 that contains material 70 without the analyte, while (c) the principle beam passes in parallel with the reference beam through a portion of region 30 that contains material 70 with the analyte. In these applications, the rotation induced by the analyte in material 70 is quantified as the difference between the rotation of the principle beam and the rotation of the reference.

In some applications of the present invention, the reference beam is not polarized, while the principle beam is polarized. In such an application, the reference beam serves as a control for estimating the portion of decrease in measured intensity of the polarized principle beam on detector 42 that is not due to polarization.

In some applications of the present invention, light source 40 comprises a monochromatic light emitting diode (LED), and detecting system 42 comprises a single photodetector. The intensity of light incident on the photodetector is proportional to the angle at which the light is rotated in the sample, and is proportional to the concentration of glucose in sampling region 30.

In some applications of the present invention, light source 40 comprises a white light emitting diode (LED) or a broad-band LED. In such an application, filter 52 comprises a filter system having (1) a polarizing filter, and (2) a tunable, linear optical filter which refracts the white light into monochromatic bands, i.e., light having various wavelengths. The filter system is typically regulated by the control unit described hereinabove. The tunable optical filter enables the emission of various wavelengths through region 30. In such an application, increasing the number of wavelengths which measure the same property within region 30 increases the signal-to-noise ratio.

For applications of the present invention in which light source 40 comprises a white LED, filter 52 typically comprises a polarizing filter. Filter 54 comprises a linear, tunable filter which sorts the polarized light from region 30 according to specific wavelength bands. In some applications of the present invention, light source 40 comprises an array of monochromatic LEDs, and each LED is adapted to transmit a specific wavelength through sampling region 30. Typically, every monochromatic LED is actuated simultaneously. Alternatively, the monochromatic LEDs are actuated in succession. In some applications of the present invention, filters 52 and 54 comprise polarizing filters. Detecting system 42 typically comprises a photodetector. Alternatively, detecting system 42 comprises an array of photodetectors, and each photodetector is configured to detect a specific wavelength band that has been emitted from a specific monochromatic LED and has traveled through sampling region 30.

In some applications of the present invention, device 20 is configured to measure the glucose concentration using photoacoustic spectroscopy. In such an application, light source 40 comprises at least one laser diode or a solid-state laser, and detecting system 42 comprises at least one acoustic detector. In some applications of the present invention, a single, tunable laser diode is configured to transmit light at variable wavelengths. In some applications of the present invention, light source 40 comprises a plurality of laser diodes. Each of the plurality of laser diodes is configured to emit a specific wavelength detectable by a single acoustic detector of detecting system 42. Alternatively, detecting system 42 comprises an array of detectors, and each detector is configured to detect a specific wavelength. As described hereinabove, filters 52 and 54 may comprise polarizing filters.

In some applications of the present invention, source 40 comprises an array of energy sources, e.g., solid-state laser sources that generate detectable photoacoustic effects. Each laser source of the array emits laser light having a respective wavelength. In such an application, detecting system 42 comprises an acoustic detector. In some applications of the present invention, source 40 comprises a plurality of solid-state lasers, and detecting system 42 comprises a plurality of acoustic detectors.

FIG. 3 shows optical measuring device 20 comprising at least one mirror 60, in accordance with some applications of the present invention. Light source 40 is configured to transmit light (as described hereinabove) into sampling region 30. Device 20 is configured to facilitate optical determination of analyte concentration within region 30 using optical methods described herein and other methods known in the art, e.g., polarimetry and/or absorbance spectroscopy. Light is reflected from mirror 60, which extends the optical path of the light within region 30. Extending the optical path of the light thus satisfies the conditions (set forth in equation 1) for optimizing the illumination/detection of analyte concentrations in the region 30. Once the light is reflected from mirror 60, it is absorbed by detecting system 42, as described hereinabove.

Detecting system 42 is shown positioned adjacent to light source 40 and on the same side of region 30 by way of illustration and not limitation. For example, light source 40 and detecting system 42 may be disposed at any suitable location with respect to region 30. Light source 40 and detecting system 42 may be physically separated by at least a portion of sampling region 30. The respective orientations of light source 40 and detecting system 42 are thus governed by the positioning of mirror 60 at any suitable location with respect to sampling region 30.

Sampling region 30 is shown comprising optically-transparent and glucose-permeable material 70 by way of illustration and not limitation. For example, sampling region 30 may comprise a suitable number (e.g., between 1 and 10) of layers of polymers, e.g., polytetrafluoroethylene (PTFE). Additionally, sampling region 30 may be surrounded by selectively-permeable membrane 31, as shown in FIG. 2.

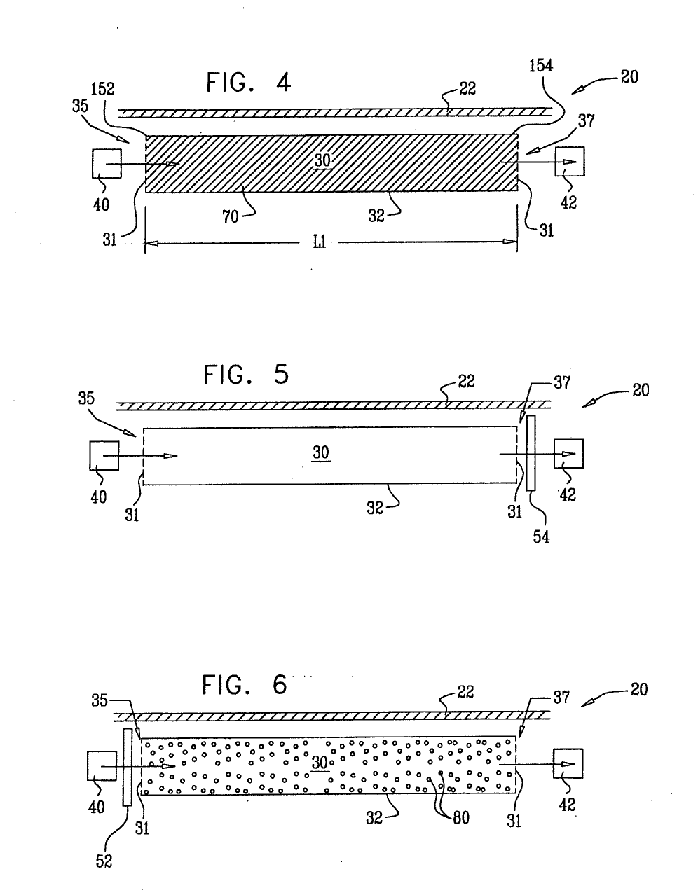

FIG. 4 is a schematic illustration of sampling region 30 as described hereinabove with reference to FIG. 1, with the exception that sampling region 30 is surrounded by a housing 32, in accordance with some applications of the present invention. In some applications of the present invention, housing 32 is shaped to define a tube, by way of illustration and not limitation. For example, housing 32 may be shaped to define a rectangular housing. As shown, sampling region 30 of housing 32 comprises optically-transparent and glucose-permeable material 70 (as described herein above with reference to FIG. 1) by way of illustration and not limitation. For example, sampling region 30 may be hollow.

Typically, housing 32 is shaped to define a substantially tubular structure having a first opening 35 and a second opening 37 to allow for passage therethrough of certain constituents (e.g., small molecules such as glucose) of interstitial fluid into housing 32. Openings 35 and 37 are shown at the portions of housing 32 that define the two longitudinal ends of housing 32 by way of illustration and not limitation. For example, first and second openings 35 and 37 may be disposed at opposing lateral sides of housing 32 that define the length of housing 32. In some applications of the present invention, openings 35 and 37 are disposed along the entire length of housing 32.

As shown, first opening 35 is disposed at a first end 152 of housing 32 and second opening 37 is disposed at a second end 154 of housing 32. Housing 32 defining sampling region 30 has suitable dimensions such that the glucose concentration within region 30 is generally in equilibrium with glucose concentrations of the interstitial fluid not within region 30.

As shown, respective membranes 31 are coupled to housing 32 at each opening 35 and 37. It is to be noted that housing 32 surrounding material 70 may be used independently of membranes 31.

Typically, due to the dimensions of housing 32, a defined volume of fluid remains within region 30 during one or more measurements of the analyte concentration in the defined volume. Since a consistent volume of fluid remains within region 30 during the one or more measurements, a lag time between successive measurements of the analyte in sampling region 30 is minimized.

It is to be noted that first and second openings 35 and 37 are shown by way of illustration and not limitation. For example, housing 32 may provide only one opening. Additionally, it is to be further noted that housing 32 is shaped to define a substantially tubular structure by way of illustration and not limitation. For example, device 20 may comprise a flat surface that defines sampling region 30.

Although device 20 is shown in FIG. 4 as not comprising filters 52 and 54, it is to be noted that filters 52 and/or 54 described herein may be used in combination with device 20 of FIG. 4.

FIG. 5 shows optical measuring device 20 as described hereinabove with reference to FIG. 4, with the exception that device 20 comprises filter 54 and housing 32 that surrounds a hollow sampling region, in accordance with some applications of the present invention.

As shown, respective membranes 31 are coupled to housing 32 at each opening 35 and 37.

In some applications of the present invention, light source 40 comprises a solid-state laser. Typically, use of the solid-state laser is configured to facilitate detection of glucose concentration by polarimetry. In such an application, detecting system 42 comprises a photodetector and filter 54 comprises a polarizing filter that is oriented perpendicularly with respect to the polarity of the laser beam as it is emitted from source 40.

Reference is now made to FIG. 6, which is a schematic illustration of device 20 as described hereinabove with reference to FIG. 5, with the exception that sampling region 30 comprises genetically-engineered cells 80 within housing 32, in accordance with some applications of the present invention. Cells 80 are genetically engineered to express a protein configured to facilitate optical quantification of the analyte in sampling region 30, e.g., as described in PCT Publication WO 06/006166 to Gross et al., and PCT Publication WO 07/110,867 to Gross et al. Cells 80 are engineered to produce a molecule (e.g., a protein, or “fluorescent material,” as described herein) that binds with an analyte and to undergo a conformational change in a detectable manner. Typically, detecting system 42 detects the conformational change, and in response, generates a signal indicative of a level of the analyte in the subject. Typically, but not necessarily, FRET techniques known in the art are used to detect the conformational change.

For applications of the present invention in which FRET is used, cells 80 are genetically engineered to produce, in situ, sensor proteins comprising a fluorescent protein donor (e.g., cyan fluorescent protein (CFP)), a fluorescent protein acceptor (e.g., yellow fluorescent protein (YFP)), and a binding protein (e.g., glucose-galactose binding protein), for the analyte. As appropriate, the sensor proteins may generally reside in the cytoplasm of cells 80 and/or may be targeted to reside on the cell membranes of cells 80, and/or may be secreted by cells 80 into sampling region 30. The sensor proteins are configured such that binding of the analyte to the binding protein changes the conformation of the sensor proteins, and thus the distance between respective donors and acceptors. It is to be noted that although CFP and YFP proteins are coupled to the analyte-binding protein, any fluorescent protein may be coupled to the analyte-binding protein.

In such an application, light source 40 comprises a source of light, e.g., a laser diode, that emits light that is absorbed by the aforementioned fluorescing molecules. Using the signal from light source 40, detecting system 42 detects the spectral changes that result from the change in distance and energy transfer from the donor to the acceptor proteins. The relative quantities of the signal resulting from subsets of the sensor proteins that are in each of the two conformations enable the calculation of the concentration of the analyte.

As shown in FIG. 6, respective selectively-permeable membranes 31 (as described hereinabove with reference to FIG. 2) are disposed at openings 35 and 37. Membranes 31 immunoisolate cells 80 and function to restrict passage of (a) cells from outside device 20 into sampling region 30, and (b) cells 80 from within sampling region 30 to outside device 20. In some applications of the present invention, portions of cells 80 are encapsulated within respective membranes within housing 32.

In some applications of the present invention, cells 80 are immobilized upon a first scaffold polymer. The polymer may be used independently of or in combination with housing 32. For example, housing 32 may surround the polymer. In some applications of the present invention, a second polymer (e.g., optically-transparent and glucose-permeable material 70 described hereinabove with reference to FIG. 1) may be used in combination with the first scaffold polymer.

In some applications of the present invention, cells 80 are not surrounded by housing 32, rather cells 80 are surrounded by a biocompatible selectively-permeable membrane. In some applications of the present invention, the membrane is configured to be optically transparent. Typically, the membrane is permeable to molecules having a molecular or characteristic weight equal to or less than the molecular weight of the analyte (e.g., glucose) configured to be measured by device 20. The membrane is configured to restrict passage of (a) cells from outside device 20 into sampling region 30, and (b) cells 80 from within sampling region 30 to outside device 20.

Alternatively, cells 80 are disposed upon a scaffold, e.g., a silicone scaffold, and portions of cells 80 are encapsulated within respective biocompatible selectively-permeable membranes. In either application, the membranes restrict passage of cells into sampling region 30 and also restrict passage of cells 80 from within sampling region 30 to outside device 20.

In some applications of the present invention, cells 80 are genetically engineered to express and secrete glucose oxidase (GOx) in-situ in region 30. Some applications of the present invention may be practiced in combination with techniques described in PCT Publication WO 06/006166 to Gross et al., and PCT Publication WO 07/110,867 to Gross et al., and in the above-cited article by Scognamiglio et al.

In some applications of the present invention, measuring glucose concentrations using FRET may be employed in combination with techniques described herein for measuring glucose using absorbance spectroscopy and/or polarimetry. Thus, combining techniques typically increases the effective signal-to-noise ratio of the device and its accuracy.

It is to be noted that the scope of the present invention includes the use of device 20 independently of cells 80, and that the proteins described herein may be disposed within sampling region 30. For example, during manufacture of device 20, genetically-engineered cells may produce the proteins described herein, which are then loaded into sampling region 30.

Alternatively or additionally, sampling region 30 comprises one or more types of microorganisms which respond to the specific analyte, e.g., glucose, in the blood of the subject, as described in U.S. Provisional Patent Application 60/588,211 to Gross et al., which is incorporated herein by reference.

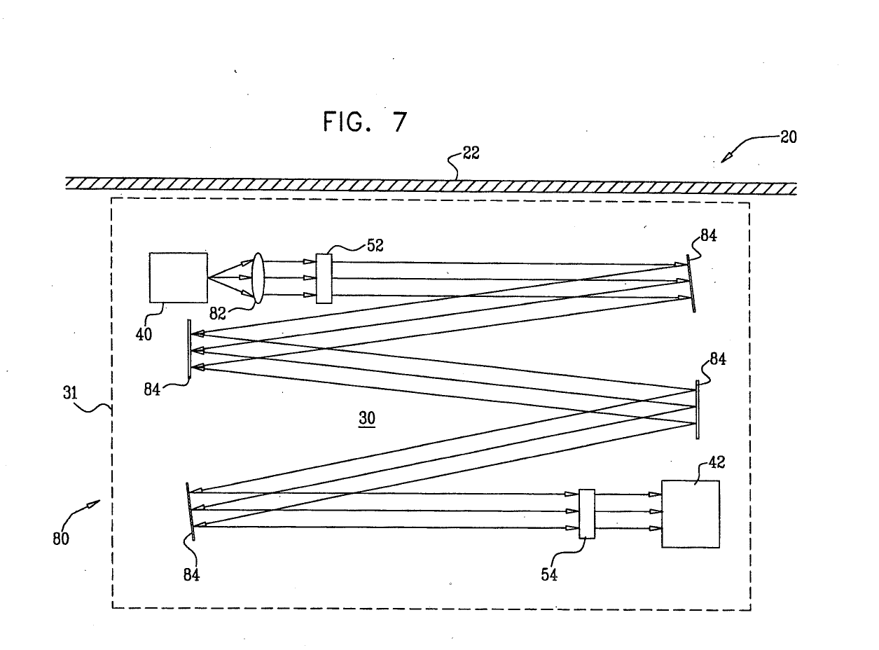

FIG. 7 is a schematic illustration of device 20 comprising a plurality of mirrors 84, in accordance with some applications of the present invention. Typically, mirrors 84 are configured to increase the length of the optical path of the light emitted by source 40. Light is reflected from mirrors 84, which extend the optical path of the light within region 30. Extending the optical path of the light thus satisfies the conditions (set forth in equation 1) for optimizing the illumination/detection of analyte concentrations in the region 30. Once the light is reflected from mirrors 84, it is passed though filter 54 and absorbed by detecting system 42, as described hereinabove.

Reference is now made to FIGS. 1-8. In the following techniques, device 20 comprises a filter (shown herein as filter 52) disposed adjacently to light source 40 (configuration not shown). Such a configuration of device 20 is applied in order to facilitate detection of glucose concentration by absorbance spectroscopy. It is to be noted that techniques described herein for measuring glucose concentration using absorbance spectroscopy comprise applying a range of wavelengths that are defined as being in the near infrared range (NIR), i.e., having wavelengths between 600 nm and 3000 nm. In some applications of the present invention, light source 40 comprises a broadband LED, and filter 52 comprises a linearly tunable filter which disperses the light from the LED into narrow wavelength bands. In such an application, detecting system 42 comprises a linear photodetector array, and each detector is positioned with respect to region 30 such that it detects a specific wavelength band.

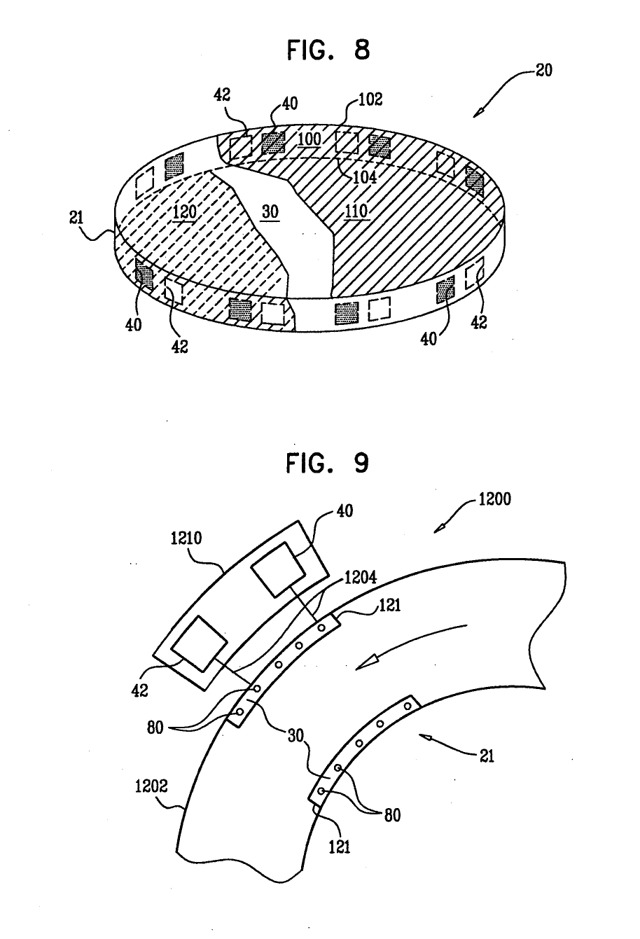

FIG. 8 shows optical measuring device 20 comprising an annular, disc-shaped support 21 defining a disc-shaped sampling region 30, in accordance with some applications of the present invention. System 20 comprises a plurality of light sources 40 and a plurality of detecting systems 42, or sensors, which are disposed circumferentially along a wall 100 of support 21. Wall 100 surrounds sampling region 30. Support 21 facilitates a suitable spatial relationship (as shown) between sampling region 30, the plurality of light sources 40, and the plurality of detecting systems 42. In this manner, light sources 40 transmit light within sampling region 30 and each detecting system 42 receives at least a portion of the transmitted light that has passed through region 30.

As shown, a plurality of pairs of adjacently disposed light sources 40 and detecting systems 42 are disposed circumferentially along wall 100 by way of illustration and not limitation. For example, the plurality of light sources 40 may be disposed successively along a first portion of wall 100, and the plurality of detecting systems 42 may be disposed successively along a second portion of wall 100 that is opposite the first portion.

In some applications of the present invention, one or more mirrors are disposed circumferentially along wall 100 of support 21. The one or more mirrors lengthen the optical path of the light emitted from the plurality of light sources (in a manner as described hereinabove with reference to FIGS. 3 and 7). Typically, the mirrors are disposed at a given geometrical orientation which optimizes the optical path length of the light transmitted from the plurality of light sources.

Support 21 has an upper surface 102 and a lower surface 104. An upper selectively-permeable membrane 110 is coupled to upper surface 102 and a lower selectively-permeable membrane 120 is coupled to lower surface 104. Typically, membranes 110 and 120 restrict passage of cells into sampling region 30. In some applications of the present invention, membranes 110 and 120 comprise hydrophobic membranes, e.g., nitrocellulose membranes. Alternatively or additionally, membranes 110 and 120 comprise polyvinylidene difluoride, or PVDF, membranes. In some applications of the present invention, membranes 110 and 120 each have a molecular weight cutoff of around 500 kDa. It is to be noted, however, that applications described herein may be implemented independently of membranes 110 and 120.

Typically, interstitial fluid passively passes through membrane 110, through sampling region 30, and finally through membrane 120. Membranes 110 and 120 provide permeability for passage therethrough of certain constituents (e.g., small molecules such as glucose) of the interstitial fluid that have a molecular weight smaller than the molecular weight cutoff defined by membranes 110 and 120. For example, the molecular weight cutoff allows passage through membranes 110 and 120 of only glucose molecules present in the interstitial fluid and of other molecules having a molecular weight less than or generally equal to the molecular weight of the glucose molecule. That is, membranes 110 and 120 are configured to restrict passage therethrough into sampling region 30 of molecules or other body fluid components having a molecular or characteristic weight substantially greater than the molecular weight of a glucose molecule, e.g., cells.

Typically, the disc-shaped sampling region 30 has an upper disc-shaped surface region (i.e., a first region exposed to the interstitial fluid) having a first surface area thereof, and a lower disc-shaped surface region (i.e., a second region exposed to the interstitial fluid) having a second surface area thereof. The first and second surface areas provide a combined large surface area for passive fluid transport through sampling region 30. In some applications of the present invention, membrane 110 is disposed in the vicinity of the upper region of sampling region 30, and membrane 120 is disposed at the lower region of sampling region 30 (configuration shown). Sampling region 30 typically comprises optically-transparent and glucose-permeable material 70 (as described hereinabove with reference to FIGS. 1-4) independently of, or in combination with, membranes 110 and 120. In some applications of the present invention, sampling region 30 comprises cells 80, as described hereinabove with reference to FIG. 6. In such an application, membranes 110 and 120 restrict passage of cells 80 from within region 30 to outside device 20.

Support 21 has a height of between about 1 mm and 2 mm (typically, between about 1.5 mm and 2 mm) and a diameter of between about 4 mm and 12 mm (typically, between about 4 mm and 6 mm). As such, the upper and lower regions of sampling region 30 each have a diameter of between about 4 mm and 12 mm (typically, between about 4 mm and 6 mm). Typically, an average total surface area of support 21 is around 69 mm̂2, and an average combined surface area of the upper and lower regions of sampling region 30 is around 39 mm̂2.

Thus, the combined surface area provided for substance transport by the upper and lower regions of sampling region 30 is typically at least 50% (e.g., at least 70%) of a total surface area of optical measuring device 20. Typically, the combined surface area provided for substance transport by the upper and lower regions of sampling region 30 is typically less than 95% (e.g., less than 90%) of a total surface area of optical measuring device 20. Typically, both the upper and lower regions of sampling region 30 allow for passive fluid transport therethrough and into sampling region 30. In some applications of the present invention, only one of the upper and lower regions of sampling region 30 allows for passive fluid transport therethrough and into sampling region 30. It is to be noted that in some applications of the present invention, FIGS. 1-3 show a lengthwise cross-section of the disc-shaped support 21 of FIG. 8, mutatis mutandis. Thus, FIGS. 1-3 may be interpreted as showing a flat, generally disc-shaped device, in which interstitial fluid flows through the top and/or bottom surfaces in each figure. Similarly, FIGS. 4-6, which show fluid flow through the left and right surfaces in each figure, may be generally disc-shaped and comprise large upper and lower surface areas for substance transport (as shown in FIG. 8).

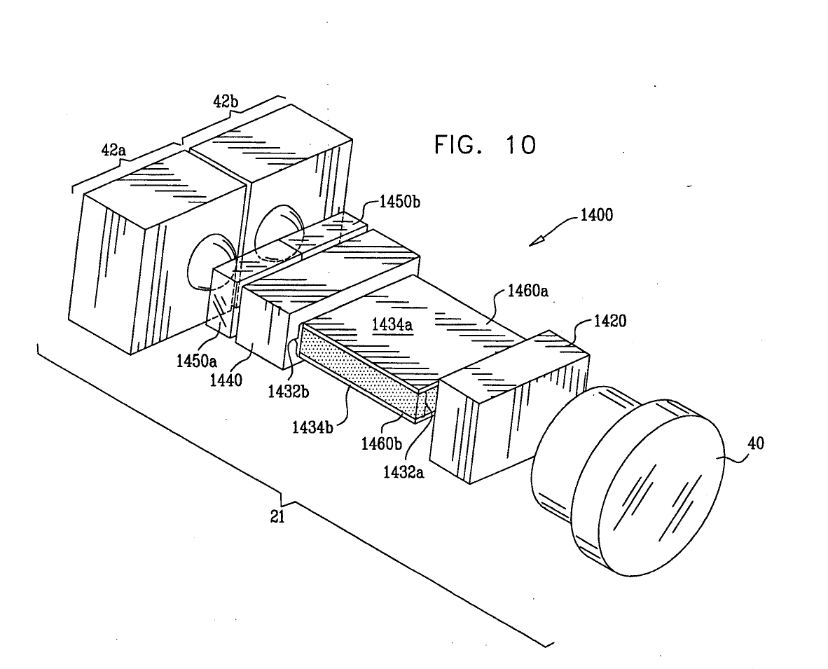

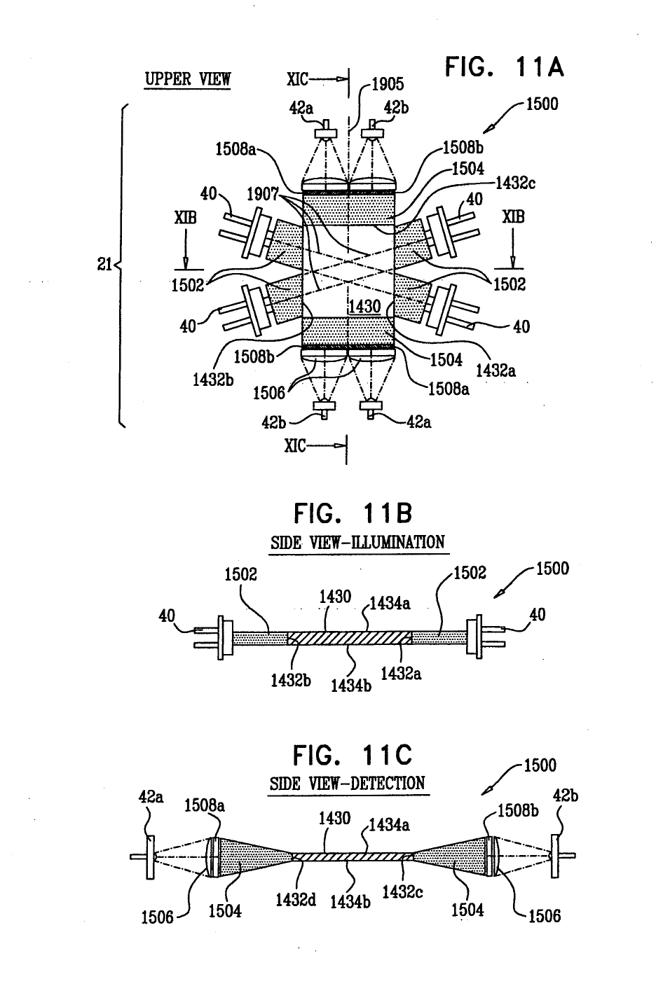

Reference is now made to FIG. 9, which is a cross-sectional schematic illustration of an optical measuring system 1200 comprising support 21 designated for implantation in a blood vessel 1202 of the subject, in accordance with some applications of the present invention. Typically, blood vessel 1202 includes a vena cava of the subject. Support 21 is shaped to define a cylindrical support 121 defining a cylindrical sampling region 30 which houses a plurality of genetically-engineered cells 80, as described hereinabove with reference to FIG. 6. In such an application, sampling region 30 is disposed in a wall of cylindrical support 121. Typically, support 121 comprises a material which immunoisolates cells 80 from cells of the body of the subject. In some applications of the present invention, support 121 is surrounded by a selectively-permeable membrane (not shown for clarity of illustration), which immunoisolates cells 80.

An electrooptical unit 1210 is disposed externally to blood vessel 1202 and houses light source 40 and detecting system 42. Unit 1210 is coupled to the support 121 via optical fibers 1204 which facilitate the propagation of light between unit 1210 and support 121.

Blood passes through vessel 1202 (in a direction as indicated by the arrow) and through a lumen defined by cylindrical support 121. As the blood passes through the lumen, components of blood are absorbed by support 121 and passes into sampling region 30. Cells 80 are engineered to produCe a molecule (e.g., a protein) that is able to bind with an analyte in the blood and to undergo a conformational change in a detectable manner. In order to measure the conformational change of the proteins, and in turn, the amount of analyte in the blood, light is provided to sampling region 30 (in a manner as described hereinabove) by light source 40 via fibers 1204. Typically, detecting system 42 detects the conformational change, and in response, generates a signal indicative of a level of the analyte in the subject. Typically, but not necessarily, FRET techniques known in the art are used to detect the conformational change.

In some applications of the present invention, cells 80 are genetically-engineered to secrete the protein into blood vessel 1202 and into the lumen defined by support 121. In such an application, the lumen of support 121 functions as the sampling region. Light travels from unit 1210 toward the lumen of support 121 and is used to detect conformational changes of the secreted proteins within the lumen of vessel 1202.

Support 21 is shown as being cylindrical by way of illustration and not limitation. For example, support 21 may comprise a flexible disc-shaped housing comprising a gel that encapsulates the cells, and is disposed in vessel 1202 in a manner which reduces the incidence of clotting in vessel 1202 and reduces fibrosis of tissue around support 21.

In some applications of the present invention, support comprises, by way of illustration and not limitation, agarose, silicone, polyethylene glycol, gelatin, an optical fiber capillary, a polymer, a co-polymer, and/or an alginate.

FIG. 10 shows an optical sensor device 1400 in which both fluorescence excitation and detection occur from respective narrow width—sides 1432a and 1432b of a substantially flat sampling region 1430, in accordance with some applications of the present invention. Flat sampling region 1430 is similar in content to sampling region 30 described hereinabove. That is, for some applications of the present invention, flat sampling region 1430 comprises the optically-transparent and glucose-permeable material, as described hereinabove with reference to FIG. 1, which describes optically-transparent and glucose-permeable material 70. For some applications, sampling region 1430 is surrounded by a selectively-permeable membrane, as described hereinabove with reference to selectively-permeable membrane 31 that surrounds sampling region 30. For other applications of the present invention, sampling region 1430 comprises genetically-engineered cells 80, as described hereinabove with reference to FIG. 6.

Excitation light that is emitted by light source unit 40 (e.g., a laser diode or any other light sources as described herein) in a given wavelength band is focused by cylindrical lens 1420 into width-side 1432a of sampling region 1430 to excite a fluorescent material disposed therein, e.g., the FRET proteins described hereinabove with reference to FIG. 6. It is to be noted that although lens 1420 is shown as being rectangular in the schematic view, lens 1420 comprises a cylindrical lens which focuses the excitation light from source toward narrow width-side 1432a of sampling region 1430. Fluorescent emitted light is collected at the opposite side of device 1400 (i.e., the side opposite light source 40) by light detecting system 42, e.g., a pair of photo-diodes, as shown by way of illustration and not limitation. A dichroic filter 1440 attenuates and filters out the transmission from sampling region 1430 of light of undesirable wavelength bands, i.e., light of wavelength bands that are different from the emission light having the wavelength bands of interest. Optical device 1400 comprises at least a pair of filters 1450 which selectively transmit light having two fluorescent wavelength bands of interest. That is, light of a respective wavelength band is filtered toward a respective light detector of detecting systems 42a and 42b.

Typically, sampling region 1430 houses fluorescent material which is excited by light having a first given wavelength band. This light is emitted from light source 40. Responsively to the excitation, the fluorescent material emits light having a second wavelength band. Typically, the emission parameters of the light emitted from the fluorescent material is proportionately affected in response to the presence of an analyte, e.g., glucose, in sampling region 1430. Thus, concentrations of the analyte in region 1430 are measured responsively to the changes in emission parameters of the light emitted by the fluorescent material.

It is to be noted that although applications described herein with reference to FIG. 10 apply fluorescence emission, any suitable light emission as described herein and any light emission known in the art, may by applied in place of the fluorescence emission.

It is to be further noted that sampling region 1430 described herein (and sampling region 30 described herein for some applications) comprises the “fluorescent material.” By way of definition, the “fluorescent material” comprises (1) an analyte-binding molecule which binds to glucose (or any other analyte) and changes its conformation upon the binding, and (2) one or more fluorescent molecules coupled to the analyte-binding material. The analyte-binding molecule is coupled to fluorescent material which fluoresces and emits light when excited by the excitation light. This emitted light is collected by detecting systems 42a and 42b which transmit intensity data which is then used (e.g., by electronics provided by the devices described herein) to calculate the level of glucose based on the amount of measured fluorescence. For some applications, the analyte-binding molecule that binds to glucose is a glucose-binding-protein, as described hereinabove which is genetically engineered to express two fluorescent molecules (i.e., a cyan fluorescent protein and a yellow fluorescent protein). Upon the binding of glucose to the glucose-binding-protein, the protein changes configuration which either draws closer or distances the two fluorescent molecules. For applications in which the fluorescent molecules are drawn closer together following the binding of glucose to the glucose-binding-protein, the following cascade of excitation and emission light provides an indication of the amount of glucose in the sampling region: (1) excitation light is emitted from light source 40 and travels toward sampling region 1430, (2) the excitation light excites a first one of the fluorescent molecules (i.e., the CFP), (3) the excited first fluorescent molecule then emits energy which excites the second of the two fluorescent molecule, and (4) the second fluorescent molecule then emits light having a wavelength band.

As shown in FIG. 10, sampling region 1430 has two large exposed flat surfaces 1434a and 1434b thereof (i.e., the upper and lower large exposed surfaces of region 1430). Surfaces 1434a and 1434b provide a large interface surface area of sampling region 1430 with tissue and/or fluid surrounding device 1400, thereby supporting optimal exchange of materials between device 1400 and its surroundings. Typically, device 1400 comprises support 21 which maintains the components in their relative spatial configurations and maintains sampling region 1430 in contact with the surrounding tissue and/or fluid. For some applications, support 21 comprises first and second selectively-permeable membranes 1460a and 1460b which surround sampling region 1430 at least in part, e.g., membrane 1460a contacts surface 1434a and membrane 1460b contacts surface 1434b. Support 21 serves as a scaffold for maintaining the fluorescent material in place within sampling region 1430 as well as for selecting the constituents, e.g., glucose, of the interstitial fluid that are exchanged between region 1430 and the surrounding tissue and/or fluid. For example, when device 1400 is implanted in the body of the subject, membranes 1460a and 1460b (1) facilitate the permeation of analytes of interest, while (2) restricting the passage therethrough of components from within region 1430 into the body which could potentially activate the host's immune system. Additionally, membranes 1460a and 1460b restrict passage therethrough of agents of the immune system from entering sampling region 1430. Support 21 and membranes 1460a and 1460b have reflective and scattering optical properties which contribute to enhancing the effectiveness of both (1) transmitting of the fluorescence excitation light and (2) directing a larger portion of the fluorescent-emitted light toward light detecting systems 42a and 42b. In such applications, light detecting systems 42a and 42b each comprise a respective lens that focuses light onto a respective active light sensing surface, e.g., silicone chip, of each detecting system 42a and 42b.

In some applications of the present invention, width—sides 1432a and 1432b of sampling region 1430 (i.e., the surfaces of region 1430 which are disposed perpendicularly to the upper and lower large exposed surfaces 1434a and 1434b of sampling region 1430, and herein referred to as “narrow width-sides”) are coated by a reflective material. This material facilitates enhancement of the effectiveness of (1) the excitation light power that passes through sampling region 1430 from light source 40, and (2) the amount of emission power that is transmitted beyond region 1430 and eventually reaches the optical detection path toward detecting system 42. Alternatively or additionally to the reflective coating, additional light sources and light detectors are optically coupled to the exposed narrow width-sides of sampling region 1430.

In addition to providing a large interface area of sampling region 1430 with the surroundings, device 1400 of FIG. 10 provides simplicity, compactness, and a slim structure that improves the ease of implantation.

FIGS. 11A-C show respective views of some applications of an optical sensor device 1500 in which the four narrow width-sides 1432a, 1432b, 1432c, and 1432b of flat sampling region 1430 are used to facilitate sample illumination and detection of fluorescent emission signal, in accordance with some applications of the present invention. The light that is produced by four light sources 40, e.g., laser diodes by way of illustration and not limitation, is guided by light guides 1502 into two opposite narrow width-sides 1432a and 1434b of sampling region 1430 in order to excite a fluorescent material that is disposed within sampling region 1430. The light that is emitted by the fluorescent material in sampling region 1430 is collected by light guides 1504 and transmitted through filters 1508 into lenses 1506 that focus the light onto light detecting systems 42, e.g., photo-diodes by way of illustration and not limitation. Device 1500 shown here is similar to device 1400, as illustrated hereinabove with reference to FIG. 10, in that two large surfaces 1434a and 1434b of sampling region 1430 are exposed to the surrounding tissue and/or fluid.

It is to be noted, as described hereinabove, that light sources 40 may comprise any light source described herein or any light source known in the art. It is to be further noted that detecting systems 42 may comprise any detecting system described herein or any detecting system known in the art. Also, as described hereinabove, region 1430 may (1) comprise any suitable medium for transport therethrough of an analyte, and (2) be surrounded by any suitable selectively-permeable membrane, as described herein.

In contrast to device 1400 as described hereinabove with reference to FIG. 10, device 1500, as described in FIGS. 11A-C provides a spatial orientation in which light sources 40 are disposed in optical communication with opposite narrow width-sides 1432a and 1432b of sampling region 1430 that are perpendicular with respect to the opposite narrow width-sides 1432c and 1432d of region 1430 that are in optical communication with light detecting systems 42. Thus, the amount of undesired light that reaches systems 42 directly from light sources 40 is significantly reduced. The homogeneity of excitation light distribution is also improved by the fact that light sources 40 are arranged on opposite narrow width-sides 1432a and 1432b of sampling region 1430 that face each other. The positioning of light sources 40 on opposing sides 1432a and 1432b of region 1430 reduces by half the distance the light from sources 40 travels toward region 1430. This mean shorter distance from light sources 40 to sampling region 1430 increases the excitation power. The positioning of detecting systems 42 on opposing sides 1432c and 1432d of region 1430 reduces by half the distance the light from region 1430 and toward detecting systems 42. This mean shorter distance from sampling region 1430 to detector systems 42 increases the efficiency, accuracy, and power of the emission signal that is detected.

Filters 1508 are arranged with respect to region 1430 in a way in which every light detecting system 42 is in optical communication with and receives light having one of the at least two fluorescence emission wavelength bands that are emitted from the fluorescent material disposed within sampling region 1430. That is, each of the two opposite sides 1432c and 1432d of sampling region 1430 that is in optical communication with light detecting systems 42 is coupled to a pair of filters 1508. Each filter 1508 filters therethrough light having a different one of the one or more emission wavelength bands emitted from the fluorescent material in sampling region 1430, and that filter 1508 transmits the band to the respective light detecting system 42 that is in optical communication therewith. In this manner, each filter 1508 restricts transmission to a respective one of detecting systems 42 of a given one of the one or more bands of spectral information from two opposite sides 1432c and 1432d of sampling region 1430. Typically, the fluorescent material in region 1430 emits at least two different spectral bands upon excitation, and the ratio of these bands is analyzed, as described hereinabove, in order to calculate the glucose concentration. Therefore, device 1500 typically provides two different types of filters 1508, wherein each filter 1508 filters therethrough a given one of the two different spectral bands.

The spatial orientation of filters 1508 and detecting systems 42 with respect to sampling region 1430 minimizes the effect of non-homogeneous distribution of the concentration of the fluorescent material within sampling region 1430 during the analysis of the data collected by systems 42. For example, in a case in which the fluorescent material within sampling region 1430 is more concentrated in given area of region 1430 (i.e., while not being evenly distributed to other areas of region 1430), a stronger fluorescent signal is emitted from that given area of region 1430 relative to other areas of region 1430. In such a case, when a respective filter 1508 is provided at both sides 1432c and 1432d for either spectral band of emission light from region 1430, the intensity ratios between the two spectral bands that are measured at either one of respective sides 1432c and 1432d of sampling region 1430 represent the actual fluorescent parameters of the sample and compensate for the uneven distribution of fluorescent material in sampling region 1430. Conversely, if only one filter 1508 was provided at either side (i.e., a first filter for filtering a first of the two bands emitted from the fluorescent material is coupled to side 1432c while a second filter for filtering a second of two bands emitted from the fluorescent material is coupled to side 1432d of sampling region 1430), this configuration does not necessarily compensate for the uneven distribution of the fluorescent material in sampling region 1430. Moreover, the size of sampling region 1430 in some applications of the present invention allows the introduction of four light sources 40 (e.g., two on each side, as shown), which enhances the excitation power of the light that reaches sampling region 1430 and facilitates even distribution of the excitation light within sampling region 1430. The relative spatial arrangement of components of device 1500 of FIGS. 11A-C supports up to four different excitation and emissions bands by providing coupling sites for (1) up to four different transmission band filters in front of up to four different light sources 40 and (2) up to four different transmission band filters, e.g., filters 1508, in front of the up to four different light detecting systems 42.

Typically, excitation light that is produced by light sources 40 is delivered, along respective excitation-light-transmission axes 1907 to two opposite narrow width-sides 1432a and 1432b of sampling region 1430 by light guides 1502. The excitation light excites the fluorescent material in sampling region 1430. Responsively, the fluorescent material emits light that is directed from sampling region 1430 toward detecting systems 42a and 42b. On its path from the fluorescent material in sampling region 1430 to detecting systems 42a and 42b, the fluorescent light is transmitted along a central light-emission-transmission axis 1905 that is at a non-zero angle with respect to axes 1907.

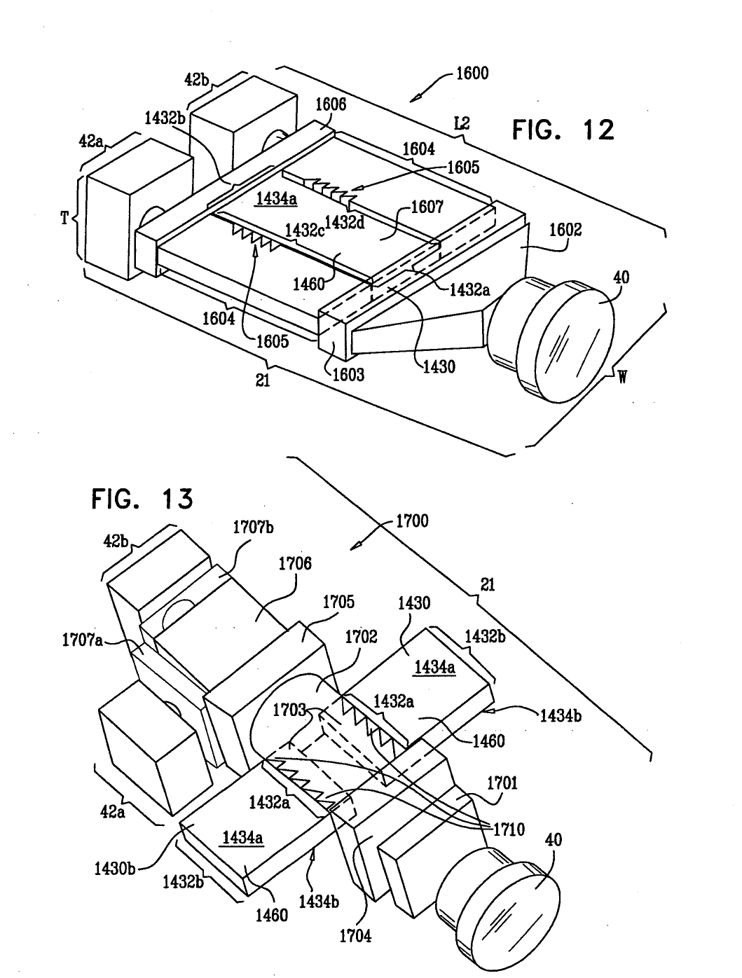

FIG. 12 shows an optical sensor device 1600 comprising a beam expander 1602 and dichroic filter 1603, in accordance with some applications of the present invention. Typically, fluorescence-excitation light produced by light source 40, e.g., laser-diode by way of illustration and not limitation, is expanded by beam expander 1602 and passes through filter 1603. Filter 1603 transmits toward sampling region 1430 the excitation wavelength band and reflects back into sampling region 1430 the fluorescence bands emitted from the fluorescent material disposed in region 1430. Accordingly, the excitation light passing through filter 1603 enters sampling region 1430 (1) directly through one of the narrow width-sides 1432a of sampling region 1430, and (2) through two other, opposing, narrow width-sides 1432c and 1432d via respective coupling-light-guides 1604 that are coupled to sides 1432c and 1432d of sampling region 1430. Prism-shaped cuts 1605 at the interface between coupling-light-guide 1604 and sampling region 1430 enhance, by local reflection, the entrance into sampling region 1430 of the amount of excitation light passing through guides 1604 from filter 1603.

Responsively to the excitation light, the fluorescent material in sampling region 1430 emits fluorescent light in all directions. Respective portions of the emitted light reach coupling-light-guides 1604, which, in turn, guide the light through one more filters 1606 onto detecting systems 42a and 42b. Typically, for applications in which detecting systems 42a and 42b each detect light having one wavelength band, two filters 1606 are provided in device 1600 which each filter light having one wavelength band to a respective detecting system 42a and 42b. Dichroic filter 1603 reflects back toward detecting systems 42 fluorescence emission light that travels from sampling region 1430 in the direction that is opposite systems 42a and 42b. Filter 1603 thus directs the light from sampling region 1430 that travels in the direction opposite detecting systems 42 which would have otherwise been lost in the absence of filter 1603. Optical device 1600 comprises a reflective coating 1607 at a portion of sampling region 1430. Coating 1607 prevents the escape from region 1430 of the excitation light as well as the fluorescent emission light from a free end (i.e., side 1432b) of sampling region 1430, thereby enhancing the effective excitation power as well as the emission power that eventually reaches detecting systems 42. Optical device 1600, as shown in FIG. 12, comprises a membrane 1460 that surrounds sampling region 1430 at least in part and serves as the interface between sampling region 1430 and the surroundings, in a manner as described hereinabove with respect to support 21 with reference to FIGS. 1 and 10.

Device 1600 of FIG. 12 provides (1) a slim structure that improve the ease of implantation, (2) a large sampling region interface to the surroundings from both large sides 1434a and 1434b of sampling region 1430, and (3) usage of at least three narrow width-sides 1432a, 1432c, and 1432d of sampling region 1430 for illumination and emission light collection. Two of these sides, i.e., sides 1432c and 1432d, serve both for illumination and collection. The spatial arrangement depicted by FIG. 12 has a length L2 of typically less than 20 mm, a width W of less than 15 mm, and a thickness T of less than 5 mm.

As shown, expander 1603 is tapered and respective lengths thereof expand in successive cross-sections thereof from light source 40 toward narrow width-side 1432a of sampling region 1430. It is to be noted that devices described herein with reference to FIGS. 1-20, may comprise expander 1603 and/or may comprise an expander disposed between sampling region 1430 and optical elements (e.g., guides, filters, and lenses) which collect the light from narrow-width side 1432 of sampling region 1430 and direct the collected light toward a respective detecting system 42. Expanders that are disposed between sampling region 1430 and the light-collecting optical elements taper in a manner in which respective lengths thereof at successive cross-sections thereof expand from narrow width-side 1432 of sampling region 1430 toward detecting systems 42.

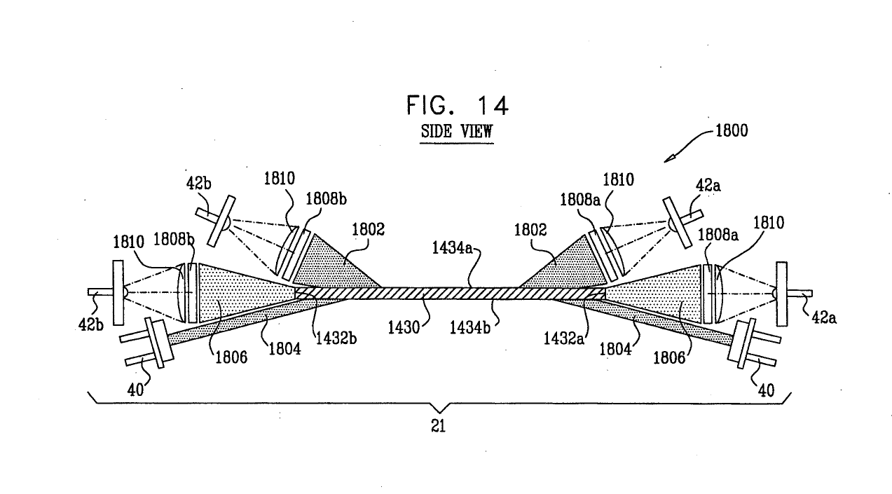

FIG. 13 shows an optical sensor device 1700 comprising a cylindrical light guide 1702 and a dichroic filter 1704, in accordance with some applications of the present invention. Excitation light that is transmitted from light source 40, e.g., laser-diode by way of illustration and not limitation, passes through a cylindrical lens 1701 that focuses light into cylindrical light guide 1702 and through dichroic filter 1704. Filter 1704 (1) filters light to a desired wavelength band of excitation light, (2) transmits the fluorescence excitation light, and (3) reflects toward detecting systems 42a and 42b the fluorescence emission light from first and second sampling regions 1430a and 1430b that are each coupled at respective portions to light guide 1702 (as described hereinabove with reference to filter 1603). Optical device 1700 comprises two sampling regions 1430a and 1430b that are each coupled to respective first and second light guides 1703a and 1703b. Each light guide 1703a and 1703b, in turn, is coupled to cylindrical light guide 1702 and serves to transmit light back and forth into the respective sampling region 1430 coupled thereto. Each sampling region 1430a and 1430b is coupled, at least in part to a respective selectively-permeable membrane 1460 which (1) serves as the interface between the sampling region and the surroundings and (2) provides, e.g., immunoisolation of the sampling region, as described hereinabove.

As shown, light guides 1703 are shaped so as to provide prism-shaped cuts 1710 at the interface between each light guide 1703 and the respective sampling region 1430 to which guide 1703 is coupled. These prism-shaped cuts 1710 enhance, by local reflection, the amount of (1) excitation light that enters sampling region 1430 from light source 40, and (2) emission light from the fluorescent material disposed within sampling regions 1430a and 1430b. Each guide 1703a and 1703b is coupled to narrow width—side 1432a of respective sampling regions 1430s and 1430b.

As described hereinabove, the at least two wavelength bands of emission light are transmitted from sampling region 1430. At a given moment, depending on the level of binding of glucose to the glucose-binding protein, the protein may fluoresce light one or more wavelength bands. Fluorescent emission light from sampling regions 1430a and 1430b reenters light guide 1702, passes through dichroic filter 1705, and reaches a dichroic beam splitter 1706. Splitter 1706 splits the light into the two emission spectral bands of interest, e.g., one spectral band is reflected into a first band filter 1707a, and the other band is transmitted into a second band filter 1707b. Ultimately, each band reaches a respective light detecting system 42a and 42b which transmit intensity data which is then used to calculate a level of glucose concentration. As described hereinabove, typically, the fluorescent material in region 1430 emits at least two different spectral bands upon excitation. Therefore, device 1700 typically provides two different types of filters 1707a and 1707b, wherein each filter 1707 filters therethrough a given one of the two different spectral bands. It is to be noted however, that device 1700, and devices described herein, may transmit light having more than one, e.g., two, three, or four, wavelength bands. Devices described herein may comprise any suitable number of filters and detecting systems.

Device 1700, as shown in FIG. 13, (1) provides a the large interface area by providing two sampling regions 1430a and 1430b (i.e., wherein each sampling region 1430a and 1430b comprises respective large exposed flat surfaces 1434a and 1434b), and (2) provides two detecting systems 42a and 42b which each measure respective split, distinct wavelength bands of a respective light beam that propagates from each one of sampling regions 1430a and 1430b. As described hereinabove, the spatial configuration of device 1700 increases the sensitivity of intensity measurements of the two detected wavelength bands of light while reducing the effect of the potentially non-homogenous distribution of fluorescent material within sampling regions 1430a and 1430b. In some applications of the present invention, the exposed edges (e.g., narrow width—sides 1432b) of sampling regions 1430a and 1430b are coated with a reflective coating which reflects back into sampling regions 1430a and 1430b light that reaches these exposed surfaces. This reflecting of the light focuses the light to a defined optical path and enhances both the effective excitation power as well as the emission intensity that is eventually directed toward detecting systems 42a and 42b. For some applications of the present invention, additional light source units 40 are directed to and are in optical communication with these exposed surfaces.

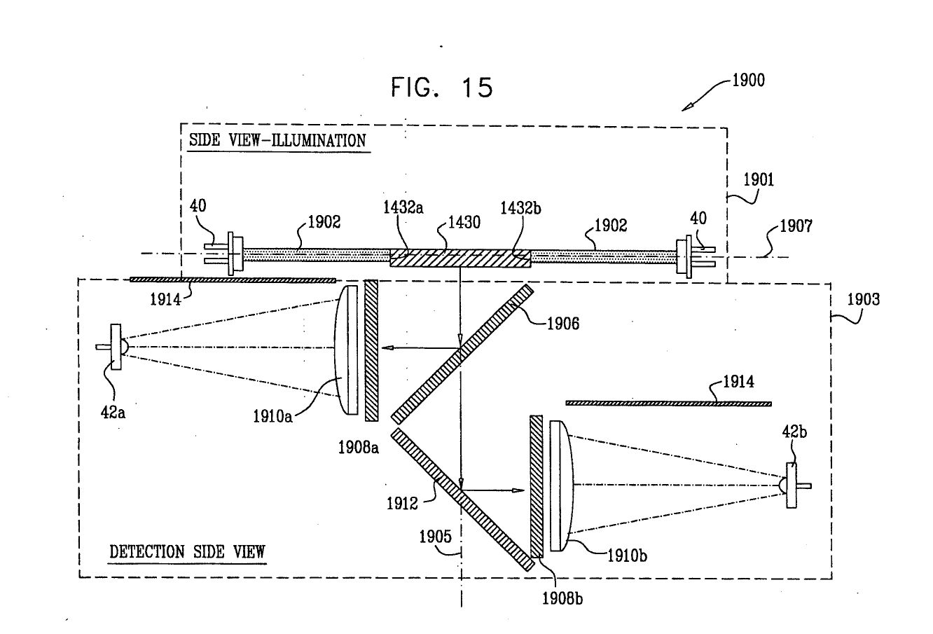

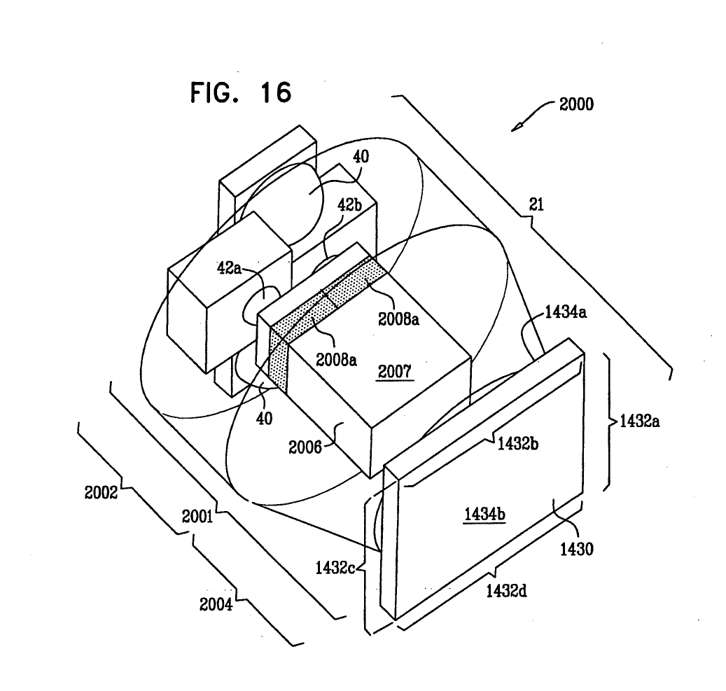

FIG. 14 shows a cross-sectional illustration of an optical sensor device 1800 that comprises one or more prisms 1802 which facilitate optimized illuminating and collecting light of the wavelength bands from a large area of sampling region 1430 (as will be described hereinbelow with reference to FIGS. 16, 17, and 19), in accordance with some applications of the present invention. Sampling region 1430 has two large exposed surfaces 1434a and 1434b (as described hereinabove with reference to FIGS. 10-13). Light is produced by six light sources 40, e.g., laser diodes (only two light sources 40 are shown for clarity of illustration). Four light sources 40 are in optical communication with narrow width-sides 1432 of sampling region 1430 (the four light sources 40 are not shown for clarity of illustration in the illustrated side-view of the device) and two light sources 40 are disposed at a non-zero angle with respect to at least a first one of large exposed surfaces 1434 of sampling region 1430 (i.e., surface 1434b, as shown).

Fluorescence excitation light is transmitted from light sources 40 to sampling region 1430 and, in turn, excites the fluorescent material (i.e., the glucose-binding protein that is coupled to the CFP and YFP proteins) in sampling region 1430. Responsively to the excitation light, the fluorescent material emits light which is then collected by four light detecting systems 42, e.g. photo-diodes. Two detecting systems 42a and 42b are in optical communication and are in parallel with opposing narrow width-sides 1432a and 1432b of sampling region 1430 (as shown), and two other detecting systems 42a and 42b are disposed at a non-zero angle with respect to at least a second one of the large exposed surfaces 1434 of sampling region 1430 (i.e., surface 1434a, as shown). Portions of light from sampling region 1430 are transmitted to respective detecting systems 42a and 42b via prisms 1802 that are in optical communication with respective portions of large exposed surface 1434a of sampling region 1430. Light guides 1804 (e.g., similar to light guides 1502, as shown in FIGS. 11A-C) are in optical communication with sampling region 1430 (e.g., with surface 1434b, as shown) and optically couple light sources 40 with sampling region 1430. Guides 1804 deliver light from sources 40 to sampling region 1430, while lateral light guides 1806 and prisms 1802 transmit light that is emitted by the fluorescent material in sampling region 1430 toward respective light detecting systems 42. Device 1800 comprises filters 1808a and 1808b which are disposed downstream from light guides 1806 and from prisms 1802. Filters 1808a and 1808b filter therethrough only the respective bands of interest which correspond to the emission bands of the fluorescent material disposed in sampling region 1430, and thereby to the amount of analyte in sampling region 1430, as described hereinabove. The amount of analyte in the body is then calculated in accordance with the amount of calculated analyte in sampling region 1430. Device 1800 comprises lenses 1810 that are each disposed between respective filters 1808 and respective detecting systems 42a and 42b. Lenses 1810 focus the light from filters 1808a and 1808b toward detecting systems 42a and 42b.

As shown, device 1800 comprises two types of detecting systems 42a and 42b which each detect and measure light having a respective wavelength band. It is to be noted that the relative spatial configuration of device 1800, as facilitated by prisms 1802 enables device to remain compact even though more than two detecting systems 42a and 42b are provided, e.g., 4, as shown. As such, device 1800 may comprise four different detecting systems 42 which each detect light having a respective wavelength band.

FIG. 15 shows an optical sensor device 1900 in which fluorescence excitation occurs from the narrow width-sides 1432a and 1432b of flat sampling region 1430, and the collection and detection of light from sampling region 1430 occurs at a different horizontal plane from the sampling region 1430, in accordance with some applications of the present invention: Device 1900 comprises an illumination portion 1901 and a detection portion 1903, and portions 1901 and 1903 are disposed along different horizontal planes of device 1900. Light detecting systems 42a and 42b are disposed at respective horizontal planes of device 1900. Device 1900 comprises a beam splitter 1906 which splits the fluorescent light into wavelength bands emitted from the fluorescent material in sampling region 1430.

Typically, excitation light that is produced by light sources 40, e.g., laser diodes, is delivered, along an excitation-light-transmission axis 1907 to two opposite narrow width-sides 1432a and 1432b of sampling region 1430 by light guides 1902. The excitation light excites the fluorescent material in sampling region 1430. Responsively, the fluorescent material emits light that is directed via device 1900 toward detecting systems 42a and 42b. On its path from the fluorescent material in sampling region 1430 to detecting systems 42a and 42b, the fluorescent light is transmitted along a central light-emission-transmission axis 1905 that is at a non-zero angle (e.g., substantially perpendicular, as shown) with respect to axis 1907. The emitted light from region 1430 is transmitted through and split by angularly-disposed dichroic beam splitter 1906 which separates the light into two wavelength bands: (1) one band is reflected by beam splitter 1906, and (2) the other band is transmitted through the beam splitter. Each band is characterized by one of the fluorescence wavelength bands of interest. That is, as described hereinabove, when excited by the excitation light transmitted to region 1430, the fluorescent material in sampling region 1430 emits at least two wavelength bands at various binding stages of each one of the analyte-binding proteins to the analyte of interest. Each band is further filtered by respective filters 1908a and 1908b in order to filter toward detecting systems 42a and 42b, respectively, light having the wavelength band of interest. Finally, field lenses 1910a and 1910b that are disposed downstream of filters 1908a and 1908b, respectively, focus the filtered light toward respective light detecting systems 42a and 42b, e.g., photo-diodes.

Optical device 1900 comprises a folding mirror 1912, which directs the light that is transmitted through beam splitter 1906, toward filter 1908b, and toward light detecting system 42b. Device 1900 comprises a compact structure by comprising folding mirror 1912 which reduces the optical path of the light and thereby reduces the overall size of device 1900. In some applications of the present invention, folding mirror 1912 is replaced by a second dichroic beam splitter which reflects the light having wavelength band to be measured toward detecting system 42b and transmits light having unwanted wavelength bands away from detecting system 42b.

In device 1900, as illustrated in FIG. 15, two intersecting planes are distinguished: (1) a horizontal plane that is parallel to large exposed surface 1434 of sampling region 1430 along which propagate the two central optical paths of light emitted from light sources 40, and (2) a vertical plane that is perpendicular with respect to the horizontal plane and projects from the center of sampling region 1430 and toward beam splitter 1906 and folding mirror 1912. These perpendicular planes define a specific optical path which significantly reduces the amount of undesirable light that enters detection portion 1903 and reaches detecting systems 42a and 42b. Additionally, dichroic beam splitter 1906 provides a filtering step, i.e., a noise-reducing step, to the final filtering performed by filters 1908a and 1908b. For applications in which device 1900 comprises a second beam splitter (i.e., in place of folding mirror 1912), as described hereinabove, a supplemental filtering step is provided along the optical path leading to detecting system 42b, thereby reducing further the noise in the optical measurements of the analyte in device 1900.

The splitting of a single light beam that is emitted by the fluorescent material in sampling region 1430 such that light having one wavelength band reaches detector 42a and light having the other wavelength band reaches detector 42b, eliminates the distorting effect typically generated by non-homogeneous distribution of the fluorescent in sampling region 1430; as described hereinabove.

It is to be noted that additional light source units 40 may be coupled to device 1900. These additional light sources 40 are typically in optical communication with and direct light toward the exposed narrow width-sides 1432 of sampling region 1430. The additional light sources 40 enhance the excitation intensity of the light transmitted toward sampling region 1430. For some applications, the additional light sources 40 add various spectral bands to the excitation spectrum provided to sampling region 1430, thereby enabling device 1900 to detect more parameters of the fluid disposed in sampling region 1430.

Detecting portion 1903 of device 1900 comprises one or more (e.g., two, as shown) blocking screens 1914 which shield at least in part the optical paths of the light that has been filtered by filters 1908 and focused by lenses 1910 toward the respective detecting systems 42a and 42b. This shielding increases the signal-to-noise ratio of device 1900 by blocking excitation light from sources 40 and by blocking light that escapes from other components of device 1900 (e.g., sampling region 1430 or light guides 1902) without first passing through beam splitter 1906 and along the optical path within detecting portion 1903, as defined hereinabove.

FIG. 16 shows an optical sensor device 2000 comprising a reflective envelope 2001 comprising a reflective cylinder 2002 and a reflective cone 2004, in accordance with respective applications of the present invention. Device 2000 comprises one or more light sources 40 (e.g., one or more LEDs) and one or more light detecting systems 42a and 42b (e.g., one or more photodiodes). Reflective envelope 2001 provides a conduit for light propagation from the one or more light sources 40 disposed at a first end of envelope 2001 (i.e., at a first end of cylinder 2002) and toward sampling region 1430 that is disposed at a second opposing end of envelope 2001 (i.e., at a second end of cone 2004). Device 2000 comprises two light sources 40, as shown: (1) one light source 40 is disposed at 12 o'clock with respect to the circular surface of the first end of cylinder 2002, and (2) the other light source 40 is disposed at 6 o'clock with respect to the circular surface of the first end of cylinder 2002. Reflective envelope 2001 enables light emitted by light sources 40 to spread evenly on large exposed surface 1434a of flat sampling region 1430. Reflective envelope 2001 facilitates emission of excitation light from light sources 40 in all directions (i.e., 360 degrees within envelope 2001).

The excitation light propagates within envelope 2001 and reaches the fluorescent material disposed within sampling region 1430. The fluorescent material coupled to the analyte is responsively excited by the excitation light, and as a result, emits fluorescent light of one or more, e.g., two, wavelength bands of interest, as described hereinabove. This emitted light is captured by a light guide 2006 that is disposed within envelope 2001. At least one external surface 2007 of light guide 2006 comprises a mirror-coating which restricts propagation into guide 2006 of stray and ambient light (e.g., the excitation light transmitted from light sources 40) from within envelope 2001. A first end of guide 2006 is in optical communication with surface 1434a of sampling region 1430, while a second, opposing end of guide 2006 is disposed in optical communication with one or more (e.g., a pair, as shown) of filters 2008a and 2008b. The distance of light guide 2006 from surface 1434a of sampling region 1430 is adjustable in a way that limits the angular content of the light that propagates from sampling region 1430 to the angles in which filters 2008a and 2008b filter the one or more spectral bands of interest (e.g., +/−20 degrees) to respective detecting systems 42a and 42b. These spectral bands correspond respectively to the wavelength of the florescence emission bands of the fluorescent material in sampling region 1430 responsively to the binding of the analyte to the molecule comprising the fluorescent material and to the excitation of the fluorescent material.

Each filter 2008a and 2008b filters a respective wavelength to a respective one of light detecting systems 42a and 42b.

The relative spatial configuration of components of device 2000 enable light to propagate generally to and from one large surface (i.e., surface 1434a) of flat sampling region 1430. In this manner, surface 1434a facilitates illumination and excitation of the fluorescent material in sampling region 1430 as well as the detection of emitted light (and thereby the amount of analyte) from the fluorescent material in sampling region 1430. Other sides of sampling region 1430 (e.g., exposed large surface 1434b, and narrow width-sides 1432a, 1432b, and 1432c) are exposed to and function as interfaces with the surroundings of device 2000.

As the excitation light is propagated toward sampling region 1430, loss of excitation energy is minimized by reflective envelope 2100 which reflects toward and spreads the light evenly on the entire surface 1434a of sampling region 1430, i.e., the large surface of sampling region 1430 that faces light sources 40.

Following the excitation of the fluorescent material in sampling region 1430 in response to the light transmitted thereto by light sources 40, light is emitted from the fluorescent material and is collected by light guide 2006. The collection of the light emitted from the entire sampling region 1430 by a single light guide 2006 (i.e., and not by a plurality of light guides, as described herein) reduces the divergent effect of the non-homogeneous distribution of fluorescent material within sampling region 1430. This effect typically distorts the actual ratio between the intensities measured in light having the different wavelength bands and the interpretation of the measurement results as discussed hereinabove with reference to FIGS. 11A-C.

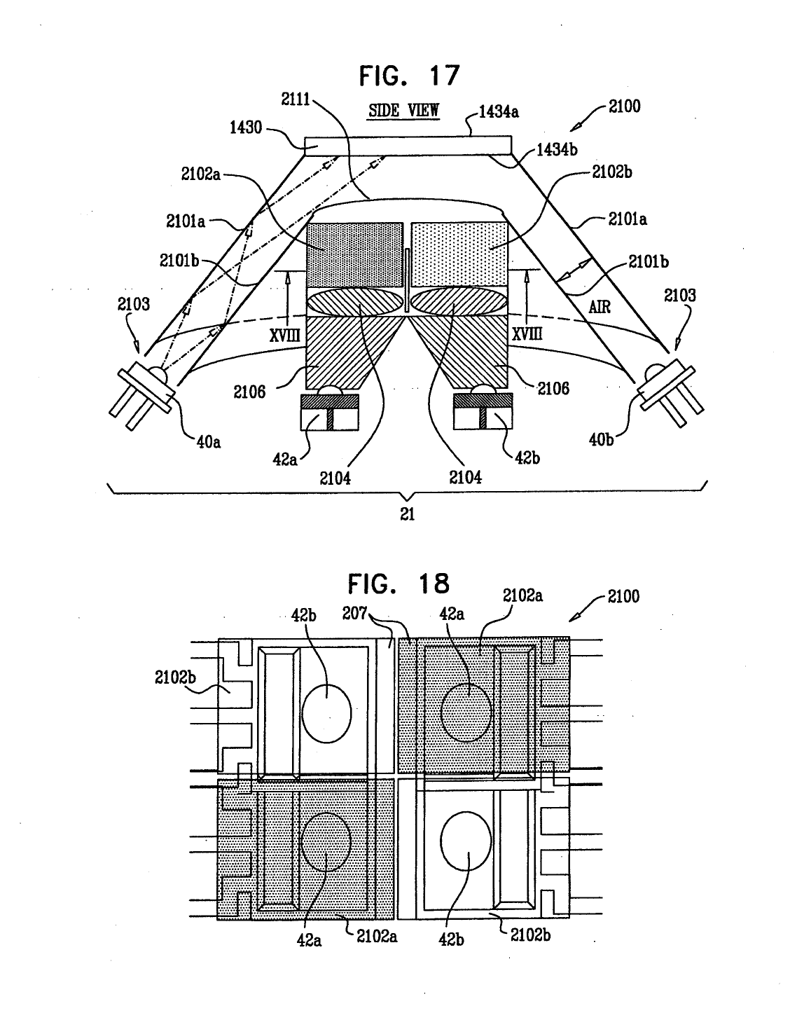

FIGS. 17 and 18 show an optical sensor device 2100 in which comprise reflective conical elements 2101a and 2101b which reflect and transmit light from light sources 40 toward sampling region 1430, in accordance with some applications of the present invention. Conical element 2101b is smaller in dimension than conical element 2101a. Conical element 2101b is therefore disposed within conical element 2101a. Such relative positioning of conical elements 2101a and 2101b creates an air-space between elements 2101a and 2101b. Conical element 2101b is shaped so as to define an upper surface 2111 that is shaped so as to provide an opening for transmission of emission light from sampling region 1430 toward detecting systems 42a and 42b.

Respective light sources 40, e.g., laser diodes, are disposed at edge 2103 of device 2100 and in the space defined by conical elements 2101a and 2101b (e.g., light source 40a is disposed at 12 o'clock, while light source 40b is disposed at 6 o'clock). That is, sampling region 1430 is disposed at the bases of both conical elements 2101a and 2101b, and light sources 40a and 40b are disposed at respective portions of edge 2103 of device 2100 that are opposite sampling region 1430.

The inner surface of conical element 2101a is reflective (e.g., the inner surface is mirror-coated), and at least the outer surface of conical element 2101b is reflective (e.g., the outer surface is mirror-coated). The fluorescence excitation light that is produced by light sources 40 is reflected back and forth between the respective reflective surfaces of conical elements 2101a and 2101b (the optical path is shown by the hatched arrows). The light propagates towards sampling region 1430 along the air-space between conical elements 2101a and 2101b. Typically, an even distribution of four light sources 40 along edge 2103 of device 2100 (i.e., at 12 o'clock, 3 o'clock, 6 o'clock, and 9 o'clock) provides generally homogeneous illuminating of large surface 1434b of sampling region 1430 that faces light sources 40.

The excitation light travels through the air-space and toward sampling region 1430 where it contacts and excited the fluorescent material of the molecule that is bound to the analyte. Responsively, the fluorescent material emits light, e.g., typically in at least two wavelength bands (as described hereinabove), that is transmitted through a series of four respective spectral band filters 2102, four respective lenses 2104, four respective light guides 2106, and finally toward four respective detecting systems 42a and 42b. Each detecting system 42a and 42b detects a different wavelength transmitted thereto by respective filters 2102a and 2102b. It is to be noted that only two of the four of the following components are shown in the cross-sectional side view of FIG. 17: light sources 40, filters 2102, lenses 2104, guides 2106, and detecting systems 42.

The emitted light from the excited fluorescent material in sampling region 1430 is propagated toward filters 2102 and ultimately toward detecting systems 42a and 42b. The filtered light from filters 2102 is focused onto detectors 42 by lenses 2104. Light guides 2106 are shaped so as to define generally trapezoidal guides. Guides 2106 have reflective inner surfaces which increase the amount of fluorescent light that is directed toward detecting systems 42 by reflecting substantial amounts of diverting light from the reflective surfaces of the light guide 2106 toward the respective light detecting system 42. For some applications, guides 2106 do not have reflective surfaces but rather reflect the light by total internal reflectance (TIR).

As shown in FIG. 18, filters 2102 comprise two pairs of filters 2102a and 2102b. Each pair of filters 2102a and 2102b transmits light having a respective one of the two fluorescence wavelength bands emitted from the fluorescent material in sampling region 1430. Filters 2102a and 2102b which filter and transmit light having the same band are positioned diagonally with respect to each other. Filter 2102a filters and transmits light having a first wavelength band to detecting system 42a, and filter 2102b filters and transmits light having a second wavelength band to detecting system 42b. This relative positioning of filters 2102a and 2102b minimizes the effect of non-homogenous distribution of the fluorescent material within sampling region 1430, as discussed hereinabove with reference to FIGS. 11A-C. That is, filters 2102a and 2102b are disposed with respect to sampling region 1430 in a manner which reduces the overall distance traveled by the light propagating from sampling region 1430 toward detecting systems 42.

Reference is now made to FIGS. 16-18. Light sources 40 of device 2000 as described hereinabove with reference to FIG. 16 typically comprise LEDs, while light sources 40 of device 2100 as described hereinabove with reference to FIGS. 17 and 18 typically comprise laser diodes. It is to be noted, however, that light sources 40 in either device 2000 and 2100 may comprise any light source described herein or any light source known in the art. Typically, the laser diodes propagate light having reduced angular dispersion of the lasers, and this in turn, reduces the loss of light intensity when the light is guided from sources 40 and toward sampling region 1430. Additionally, the laser diodes propagate light having a smaller inherent spectral band which minimizes the use of spectral band filters in the illumination path from light sources 40 to sampling region 1430. That is, for applications in which light sources 40 comprise laser diodes, the laser diodes may propagate light having only one or two wavelength bands.

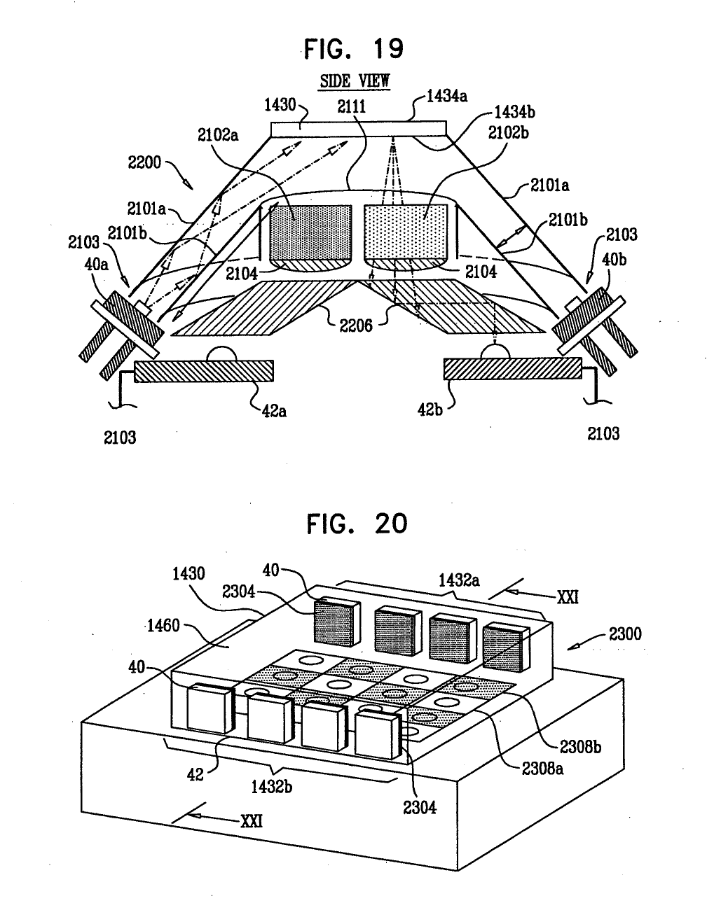

Reference is now made to FIG. 19, which is a schematic illustration of an optical device 2200 that is similar to device 2100 as described hereinabove with reference to FIGS. 17-18, with the exception that optical guides 2206 of FIG. 19 comprise rhomboid-shaped light guides 2206, in accordance with some applications of the present invention. Rhomboid light guides 2206 comprise reflective surfaces which optically align the center of the optical axes of lenses 2104 with the center of the optical axes of the respective detecting systems 42, as shown by the hatched arrows projecting from lens 2104 toward system 42. For some applications, guides 2206 do not have reflective surfaces but rather reflect the light by total internal reflectance (TIR).

Such a technique of optically aligning optical paths along the central axes of lenses 2104 and detecting systems 42 is generally applicable in order to overcome technical difficulties that may arise from the mechanical dimensions of the available components of the device. Such a configuration of the optics of device 2200 (1) increases the power of the light having the respective wavelength bands that reaches detecting systems 42a and 42b, (2) provides a circularly symmetrical angular distribution of light around each detecting system 42, and (3) thereby minimizes and perhaps eliminates the spectral shift in the transmission of light from filters 2102 which would otherwise arise from the non-zero angles of incidence of light upon detecting system 42 in the absence of rhomboidal light guides 2206. The spectral shift typically grows as the angle of incident of light grows; therefore, the relative spatial orientation of the optics of device 2200 prevents the angle of incidence upon detecting systems from shifting from approximately 90 degrees. As an alternative to rhomboid guides 2206, light beam re-direction and folding can be achieved by other optical components, e.g., prisms, mirrors, or beam splitters.

FIGS. 20-22 show an optical sensor device 2300 comprising one or more arrays of light sources 40 and a detector array of detecting systems 42, in accordance with some applications of the present invention. Device 2300 comprises a sampling region 1430 and two linear arrays of light sources 40, e.g., surface-emitting lasers or LEDs, that are disposed on opposite narrow width-sides 1432a and 1432b of sampling region 1430 and face each other. Device 2300 further comprises detector array, e.g., a CMOS detector array comprising a plurality of detecting systems 42a and 42b. Light that is produced by light sources 40 reaches the substantially flat sampling region 1430 that fills the space between the arrays of light sources 40. Typically, as shown, a selectively-permeable membrane 1460 is disposed at large exposed surface 1434a of sampling region 1430, as described hereinabove. Membrane 1460 maintains in place the fluorescent material in sampling region 1430, while also immunoisolating device 2300 and allowing exchange of material with the areas surrounding device 2300, as described herein.

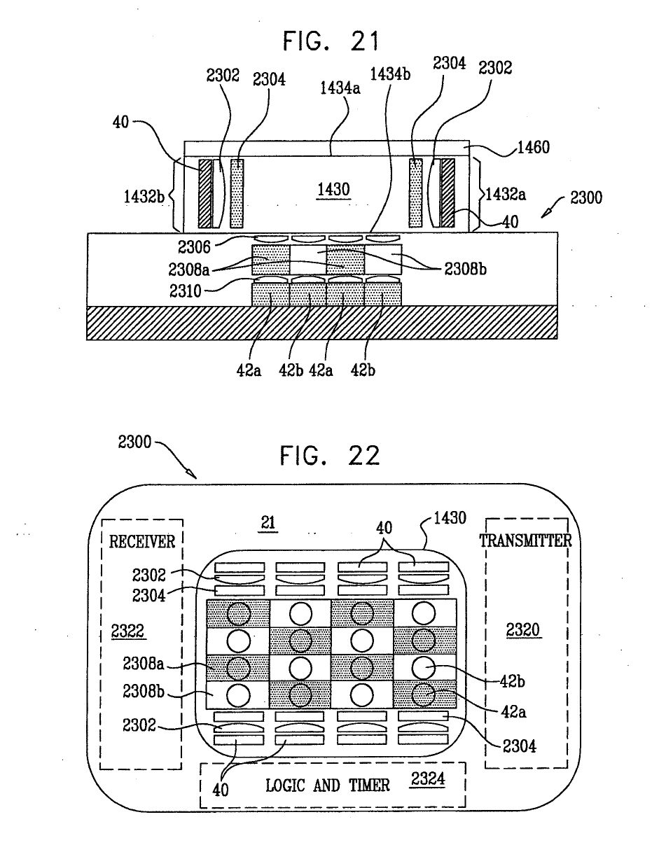

FIG. 21 shows a cross-sectional side view of device 2300, and FIG. 22 shows a top-view of device 2300. As shown in FIG. 22, device 2300 provides electronics which comprises a transmitter 2320, a receiver 2322, and a logic and timer 2324. These electronics facilitate the transmission of energy in device 2300 and the transmission of information collected from sampling region 1430 by detecting systems 42. This information is then calculated by device 2300 in order to determine the concentration of glucose in sampling region 1430, and thereby in the body of the subject. It is to be noted that devices described herein with reference to FIGS. 1-22 comprise the electronics as shown in FIG. 22.

Light from each light source 40 is focused via a respective lens 2303 to a dichroic filter 2304 which filters therethrough light having a desired excitation wavelength band from light source 40 and into sampling region 1430. Lenses 2303 and filters 2304 are arranged with respect to device 2300 in respective arrays.

Once the fluorescent material in sampling region 1430 is excited by the excitation bands, fluorescent light is typically emitted in all directions from the fluorescent material in sampling region 1430. The emitted light from the fluorescent material passes through: (1) a first micro-lens array 2306 which focuses the emitted light toward an array of filters 2308a and 2308b, and (2) the array of filters 2308a and 2308b. The emitted light from region 1430 is then filtered by filters 2308a and 2308b into respective wavelength bands of interest. These bands are then propagated through a respective lens of a second micro-lens array 2310 before reaching respective detecting systems 42a and 42b.

Device 2300 is designed and assembled in a way that the optical properties of each lens 2302, and the distances between lenses 2302, filters 2304, and detecting systems 42a and 42b are such that each detector 42 receives light that has been transmitted through a single filter 2308 in an angular content that is the appropriate one for the respective filter 2308. Each filter 2308a and 2308b is configured so as to transmit one of the two wavelength emission bands of interest to a respective detecting system 42a and 42b.

Filters 2308a and 2308b are arranged in a pattern such that nearest-neighbor (NN) filters are not of the same kind. For example, as shown in FIG. 20, filters 2308a and 2308b which filter and transmit the same wavelengths are arranged diagonally with respect to each other, thereby creating a checkerboard pattern of the filters. Additionally, the size of each filter 2308a and 2308b is small relative to the spatial variations in and non-homogeneous distribution of the fluorescent material concentration within sampling region 1430. Thus, the size and relative distribution of filters 2308a and 2308b enable the calculated mean ratio between measured intensities of light of a particular wavelength band propagating through every NN pairs of filters 2308a and 2308b corresponds to the mean ratio that is typically measured on a single fluorescent molecule of the fluorescent material.

For some applications, light sources 40 comprise laser diodes. For other applications, light sources 40 comprise LED units. For applications in which light sources 40 comprise LEDs, respective filters 2304 are disposed in the optical transmission path of each LED (as shown in FIG. 20) in order to select light having a fluorescence excitation wavelength band and avoid the infiltration into sampling region 1430 of light having wavelength bands that are similar to the fluorescent emission wavelength bands transmitted from the excited fluorescent material in region 1430. For some applications in which light sources 40 of device 2300 comprise LEDs, a single lens, a lens array, and/or one or more light guides could be added in the illumination path of the light sources 40 in order minimize excitation energy loss on path from source 40 to sampling region 1430. These added components also control the angular content of the beams transmitted to region 1430 so that light reaches filters 2308a and 2308b within an appropriate range of angles, thereby ensuring appropriate wavelength transmission for applications in which filters 2308a and 2308b comprise interference band filters.

Reference is now made to FIGS. 1-22. For some applications, detecting systems 42a and 42b comprises CMOS sensors by way of illustration and not limitation. For example, detecting systems 42a and 42b may comprise a charge-coupled device (CCD), electron multiplying CCD (EMCCD), intensified CCD (ICCD), and/or electron bombardment CCD (EBCCD). CCD technology-based devices yield very low reading variations among the detectors (pixels) under equal signal, and are typically more sensitive. CMOS technology-based devices, are typically more cost-effective than CCD-based devices, for the presented application.

Alternatively or additionally to lenses 2302, a screen shaped to define a pinhole having adjustable sizes and thicknesses may be disposed in the optical path of illumination from light source 40 in order to limit the angular content of light transmitted to sampling region 1430. These holes minimize the transmission into sampling region of stray light and light of an undesired propagation angle. However, such holes may yield a higher loss of signal intensity than the intensity of light transmitted through lenses 2302 independently of the pinholes.

Device 2300 provides a large sample-region-to-surroundings interface area in a very slim device. The short optical distance from sampling region 1430 to the array of detectors 42a and 42b reduces the need for condensing optics on the illumination path and for collection optics in the detection path. Additionally, such a configuration helps ensure that non-homogeneities in fluorescent material spatial distributions are overcome by averaging the detected wavelength band intensities of light by the electronics coupled to detecting system 42a and 42b. Production technologies involved in producing device 2300 are generally similar to production technologies used in producing semiconductors, which facilitate miniaturization, precision, cleanness, and cost-effective mass-production.

In general, for all the aforementioned applications of the present invention, the optical elements that are used are known to those skilled in the art and are designed for optimal performance by well known design methods and simulation tools. Accordingly, all light guides and lenses are shaped to transmit the optimal amount of light at the appropriate angles for illumination and for signal collection, respectively. The bulk material and the coating of the light guides are adjustable for maximal transmission and total internal reflection. The surface of the light guides is either surrounded by air, low-refractive index material, or, coated by highly reflective material, depending on the housing of the system.

Reference is now made to FIGS. 1-22. It is to be noted that sampling regions 30 and 1430 described herein may comprise genetically-engineered cells 80, as described hereinabove with reference to FIG. 6. For some applications, the cells produce and secrete a molecule that binds to the analyte and comprises a fluorescent protein into the sampling region. For other applications, the devices do not comprise cells 80 and comprise only the fluorescent proteins in the sampling region. Sampling regions 30 and 1430 are shaped so as to provide a total surface area for transfer of fluids to and from the area surrounding the devices described herein is between 10 and 100 mm̂2 or between 100 and 700 mm̂2, e.g., 20 mm̂2 and a volume of between 10 to 1000 mm̂3 or between 1000 and 10000 mm̂3, e.g., 100 mm̂3. These parameters are selected such that the ratio of the volume expressed in cubic millimeters to the surface area expressed in square millimeters is between 1 and 14 mm, e.g., between 2 and 8 mm.

Reference is again made to FIGS. 1-22. It is to be noted that light source 40 may comprise any light source known in the art, and specifically those mentioned herein, e.g., an LED or laser diode. For applications in which a laser diode is coupled to the devices described herein, the laser diode may transmit light having a narrow spectral band and low inherent angular dispersion. The property of the light as having a narrow spectral band reduces, or fully eliminates, the need for filters in the illumination path. The property of the light as having a low inherent angular dispersion reduces loss of light and minimizes the need for condensing optics. This property is particularly advantageous when sampling regions 30 and 1430 described herein comprise generally flat sampling regions. Conversely, when light source 40 comprises an array of LEDs having a wide intrinsic spectral band, a wide absorption spectrum is provided to the fluorescent material in the sampling regions. In any case, for applications in which the devices described herein comprise one or more LEDs, the devices typically comprise one or more filters and/or one or more condensing optics between LED light source 40 and sampling region 30 or 1430 in order to filter the excitation light prior to the light reaching the fluorescent material in sampling region 30 or 1430. Alternatively, for applications in which the devices described herein comprise laser diodes (which transmit narrow spectral bands toward the sampling region), fewer or no filters or condensing optics are disposed between light source 40 and sampling region 30 or 1430.

The choice of whether to use a laser diode or a LED depends, in addition to the aforementioned considerations, on their availability to produce light in the desired optical spectrum and on the relative cost-effectiveness of the choice. Moreover, additional light source types may be found optimal in view of the aforementioned consideration for a specific application without changing the principles of the described applications of the invention and remaining within the scope of this invention. Such light source types comprise, for example, organic light emitting diodes (OLED), surface emitting lasers, and/or solid state lasers, etc. For applications in which light sources 40 comprise LEDs, typically, filters are disposed in the optical transmission path of each LED in order to select light having a fluorescence excitation wavelength band and avoid the infiltration into the sampling region of light having wavelength bands that are similar to the fluorescent emission wavelength bands of light transmitted from the excited fluorescent material in the sampling region.

The optical sensor devices, as described herein, may also be configured as a dual or multi-detector, with more than one light source 40 and more than one or more than one pair of photo-detectors (i.e., more than one detecting system 42). For applications in which more than one light source 40 is coupled to the devices described herein, more than one spectral band may be transmitted from the device, e.g., each light source transmits a respective band. In such an application, the device provides additional fluorescent indicators that are capable of detecting additional analytes. Light of the same excitation wavelength band may excite different fluorescence wavelengths in fluorescent molecules of different types. Alternatively, multiple light source units may be used to excite different fluorescent molecules, wherein each light source unit emits light of a different excitation wavelength band causing light of different emission wavelength band of fluorescence response. Use of multiple light sources may also be applicable for adding spectral points, i.e., more equations in the results analysis, without increasing the number of unknowns. Moreover, additional light sources and light detecting systems serve to enhance excitation power and detection sensitivity, respectively, hence enhance the signal to noise ratio (SNR).

In order to increase the resistance and durability of the optical devices described in the various applications of this invention, for some applications of the present invention, a filler is disposed in the spaces between the components of the devices. This filler typically comprises an optically transparent material that reduces or eliminates humidity and liquid infiltration. The filler material has a low refractive index which maintains the optical properties of the elements that are described in the different applications of the present invention that were designed while considering the medium between every component of the devices described herein. Accordingly, the optical components specifications may be adjusted with respect to the choice of filler material.

The filler may comprise any suitable polymer known in the art, e.g., epoxy, silicone, and/or parylene. For some applications, the spaces between the components of the devices described herein may be filled with silicone in liquid form. Combinations of part or all of the aforementioned materials could also be considered in certain designs.

Reference is again made to FIGS. 1-22. Light sources 40 may be configured to emit light having more than one, e.g., two or more wavelength bands of excitation light toward sampling regions 30 and 1430.

The longest dimension of the devices that are described in aforementioned applications and depicted in FIGS. 1-22 may range from the maximal order of magnitude of 40 mm, when using off-shelf components, to 5 mm, when producing customized components.

It is to be further noted that the scope of the present invention includes the use of any of the optical sensing devices (independently or in combination) described with respect to FIGS. 1-22 as implantable sensors (e.g., subcutaneously-implantable or otherwise) for measuring the concentration of a particular analyte in any fluid of a body of a subject.

Reference is again made to FIGS. 1-22. The scope of the present invention includes the use of genetically engineered cells in the sampling region which are genetically engineered to produce the fluorescent material, as described in PCT Publication WO 06/006166 to Gross et al., and PCT Publication WO 07/110,867 to Gross et al.

Reference is now made to FIGS. 1, 3, 4, 8, and 9. In some applications of the present invention, device 20 and system 1200 lack filters 52 and 54, and techniques are applied in order to facilitate detection of glucose concentration by absorbance spectroscopy. In some applications of the present invention, light source 40 comprises an array of narrow-band LEDs, and detecting system 42 comprises a photodetector. In some applications of the present invention, light source 40 comprises a tunable laser diode, and detecting system 42 comprises a photodetector.

It is to be noted that sampling regions 30 and 1430 described herein may have any suitable length in accordance with a level of specificity and sensitivity of the devices described herein. Increasing the length of region 30 increases the optical path length of the light, thereby increasing the sensitivity of the devices described herein.

Reference is made to FIGS. 1-22. It is to be noted that for techniques by which glucose concentration is measured using absorbance spectroscopy, some scattering of light may occur in response to light deflecting from components disposed within the interstitial fluid. In some applications of the present invention, the scattering induced by the absorbance spectroscopy comprises Raman scattering, which is observed when monochromatic light is incident upon optically-transparent (negligible absorption) media. In addition to the transmitted light, a portion of the light is scattered. Therefore, for some applications of the present invention, device 20 comprises any suitable number of detectors which may be positioned at various locations with respect to device 20 in addition to detecting system 42, which is typically disposed in the optical path of the emitted beam. For example, detectors may be positioned in parallel and/or perpendicular orientations with the optical path (as defined by the arrows in each figure) of the emitted beam. Such a configuration of detectors enhances the signal to noise ratio of the measurement of glucose concentrations by device 20. In such an application, light source 40 emits light in the near infrared (NIR) range, e.g., between 600 nm and 1000 nm.

Reference is now made to FIGS. 2, 6, and 7. A modulator may be added to filter 52 (adjacent to light source 40), to cause the polarization of the light to vary by a given angle. In some applications of the present invention, the modulator comprises a Faraday rotator. In some applications of the present invention, the modulator comprises a single Pockel's electro-optic effect modulator. In some applications of the present invention, a closed-loop system using a Pockel's cell is used with a multiwavelength light source. In such an application, the modulator may compensate for unwanted depolarization of the light within region 30. In some applications of the present invention, the modulator comprises a liquid crystal based rotator in order to modulate the azimuth of the linearly polarized light emitted from source 40.

Reference is further made to FIGS. 1-22. In some applications of the present invention, devices and systems described herein comprise a transmitter and a receiver. The transmitter is configured to be disposed in communication with detecting system 42, and the receiver is configured to be disposed remotely, e.g., outside the body of the subject. Typically, following the measurement of a parameter of the analyte in sampling regions 30 and 1430, the transmitter transmits to the receiver an indication of the measured parameter. For applications in which the receiver is disposed outside the body of the subject, the receiver may notify the subject of the parameter in a humanly-perceptible manner. For example, the receiver may comprise a watch worn by the subject, and the watch may be configured to display the measured parameter on a display.

It is to be noted that the scope of the present invention includes the use of any of the optical sensing devices (independently or in combination) described with respect to FIGS. 1-22 for sensing constituents of fluids other than glucose. For example, apparatus described herein may be used to detect levels of calcium ions present in the fluid of the subject, mutatis mutandis. It is to be yet further noted that devices described herein may be used outside of a body of a subject, and may be used to detect constituents of fluid outside a body of a subject.

It is to be further noted that the scope of the present invention includes the use of any of the optical sensing devices (independently or in combination) described with respect to FIGS. 1-22 for measuring the concentration of a particular analyte in any fluid of the body of the subject.

The scope of the present invention includes applications described in one or more of the following:
U.S. patent application Ser. No. 11/632,587 to Gross et al., which is the US national phase application of PCT Patent Publication WO 06/006166 to Gross et al., entitled “Implantable power sources and sensors,” filed Jul. 13, 2005;
U.S. patent application Ser. No. 12/225,749 to Gross et al., which is the US national phase application of PCT Patent Publication WO 2007/110867 to Gross, entitled “Implantable sensor,” filed Mar. 28, 2007;
U.S. patent application Ser. No. 12/344,103 to Gross et al., entitled, “Implantable optical glucose sensing,” filed Dec. 24, 2008; and/or
U.S. Provisional Patent Application 61/149,110 to Gil et al., entitled, “Compact optical sensor for flat fluorescent sample regions,” filed Feb. 2, 2009.

All of these applications are incorporated herein by reference.

For some applications of the present invention, techniques described herein are practiced in combination with techniques described in one or more of the references cited in the Background section and the Cross-references section of the present patent application. All references cited herein, including patents, patent applications, and articles, are incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
a support configured to be implanted within a body of a subject;
a sampling region coupled to the support, the apparatus configured to passively allow passage through the sampling region of at least a portion of fluid from the subject; and
an implantable optical measuring device in optical communication with the sampling region, comprising:
at least one light source configured to transmit light through at least a portion of the fluid, and
at least one sensor configured to measure a parameter of the fluid by detecting light passing through the fluid.

2.-6. (canceled)

7. The apparatus according to claim 1, wherein a ratio of (a) a volume of the sampling region expressed in cubic millimeters to (b) a surface area of the sampling region expressed in square millimeters, is between 1 and 14 mm.

8. The apparatus according to claim 7, wherein the ratio of (a) the volume of the sampling region expressed in cubic millimeters to (b) the surface area of the sampling region expressed in square millimeters, is between 2 and 8 mm.

9.-17. (canceled)

18. The apparatus according to claim 1, wherein the at least one light source comprises a plurality of light sources, and wherein the at least one sensor comprises a plurality of photodetectors.

19. (canceled)

20. The apparatus according to claim 1, wherein:
the support is shaped to define a wall surrounding the sampling region,
the at least one light source comprises a plurality of light sources disposed along the wall of the support and configured to transmit light through the sampling region, and
the at least one sensor comprises a plurality of sensors disposed along the wall of the support and configured to receive at least a portion of the light passing through the fluid.

21. The apparatus according to claim 1, wherein the light source and the sampling region are disposed at a first horizontal plane of the device, and the at least one sensor is disposed at a second horizontal plane of the device.

22. The apparatus according to claim 1, wherein the light source is configured to transmit the light to the sampling region from a direction that is at a non-zero angle with respect to a direction of a central axis of a light beam that originates in the sampling region and propagates toward the at least one sensor.

23. The apparatus according to claim 22, wherein the light source is configured to transmit the light to the sampling region from the direction that is substantially perpendicular to the direction of the central axis of the light beam that originates in the sampling region and propagates toward the at least one sensor.

24.-29. (canceled)

30. The apparatus according to claim 1, wherein the support has a first surface and a second surface, and wherein the apparatus further comprises:
a first selectively-permeable membrane coupled to the first surface; and
a second selectively permeable membrane coupled to the second surface.

31. The apparatus according to claim 1, wherein:
the fluid includes components of blood of the subject,
the support is configured for implantation within a blood vessel of the subject, and
the apparatus is configured to facilitate a measurement of a parameter of blood of the subject.

32. The apparatus according to claim 31, wherein the blood vessel includes a vena cava of the subject, and wherein the support is configured for implantation within the vena cava of the subject.

33. The apparatus according to claim 31, wherein the optical measuring device is configured to be disposed externally to the blood vessel, and wherein the optical measuring device is configured to be in optical communication with a vicinity of the blood vessel in which the support is implanted.

34. The apparatus according to claim 31, wherein the support is shaped to define a cylindrical support, the cylindrical support defining a lumen thereof that surrounds the sampling region.

35. (canceled)

36. The apparatus according to claim 31, wherein the parameter of the blood includes a level of glucose in the blood, and wherein the apparatus is configured to facilitate a measurement of the level of glucose in the blood of the subject.

37. The apparatus according to claim 1, wherein the apparatus further comprises a tunable filter configured to refract the light emitted from the light source into a plurality of monochromatic bands.

38. (canceled)

39. The apparatus according to claim 37, wherein the sensor comprises a plurality of photodetectors, each photodetector being configured to detect a respective one of the plurality of monochromatic bands.

40.-41. (canceled)

42. The apparatus according to claim 1, wherein the sampling region has at least one surface thereof configured for the passage of the portion of fluid therethrough, the surface having a surface area that is at least 50% of a total surface area of the apparatus.

43. The apparatus according to claim 1, wherein the sampling region has at least one surface thereof configured for the passage of the portion of fluid therethrough, the surface having a surface area that is at least 70% of a total surface area of the apparatus.

44. The apparatus according to claim 1, wherein the sampling region has a length between 1 mm and 10 mm.

45. The apparatus according to claim 1, wherein the sampling region has a length between 10 mm and 100 mm.

46. (canceled)

47. The apparatus according to claim 1, wherein the light source and the sensor are physically separated by at least a portion of the sampling region.

48.-63. (canceled)

64. The apparatus according to claim 1, wherein the light source is configured to emit polarized light, and wherein the apparatus further comprises at least one first polarizing filter having an orientation thereof and configured to filter the polarized light emitted from the light source into the sampling region.

65. The apparatus according to claim 64, further comprising at least one second polarizing filter configured to filter to the sensor the polarized light passing through the sampling region.

66. The apparatus according to claim 65, wherein the second polarizing filter has an orientation thereof that is substantially perpendicular to the orientation of the first polarizing filter.

67. The apparatus according to claim 64, wherein the light includes visible light, and wherein the apparatus further comprises a tunable filter configured to refract the light emitted from the light source into a plurality of monochromatic bands.

68.-72. (canceled)

73. The apparatus according to claim 1, further comprising a selectively-permeable membrane coupled to the support, the membrane being configured to surround the sampling region.

74.-108. (canceled)

109. The apparatus according to claim 1, further comprising one or more folding optical elements coupled to the support and configured to reduce at least one physical dimension of the apparatus, wherein the one or more folding optical element is selected from the group consisting of: a mirror, a rhomboid-shaped element, a prism-shaped element, and a beam splitter.

110.-142. (canceled)

143. The apparatus according to claim 1, wherein the light source is configured to transmit light toward the sampling region in a direction that is at a non-zero angle with respect to a vector of passage of the fluid into the sampling region.
